(19) United States
(12) Patent Application Publication
(10) Pub. No.: US 2025/0101476 A1
(43) Pub. Date: Mar. 27, 2025

(54) SYNTHETIC BIOLOGY APPROACH TO SYNTHESIZE NICOTINIC ACID FROM 3-PICOLINE

(71) Applicant: KCAT ENZYMATIC PRIVATE LIMITED, Bengaluru (IN)

(72) Inventors: Pravin Kumar R, Bengaluru (IN); Gladstone Sigamani G, Bengaluru (IN); Roopa L, Bengaluru (IN); Anuj J. Shetty, Bengaluru (IN); Pooja M N, Bengaluru (IN); Rajashekar Jarubandi, Bengaluru (IN); Dipa Natarajan, Bengaluru (IN)

(73) Assignee: KCAT ENZYMATIC PRIVATE LIMITED, Bengaluru (IN)

(21) Appl. No.: 18/894,022

(22) Filed: Sep. 24, 2024

(30) Foreign Application Priority Data
Sep. 26, 2023 (IN) 202341064550

(51) Int. Cl. C12P 17/12; C12N 9/02; C12N 9/04; C12N 15/70; G16B 10/00; G16B 15/00

(52) U.S. Cl. CPC C12P 17/12; C12N 9/0004; C12N 9/0006; C12N 9/0008; C12N 9/0077; C12N 15/70; G16B 10/00; C12Y 102/01007; C12Y 114/15; G16B 15/00

(57) ABSTRACT

The present invention provides a method for synthesizing nicotinic acid from 3-picoline using transformed recombinant host cells with synthetically designed gene constructs as whole cell biocatalysts. Adaptive engineering of aromatic ring metabolizing genes isolated from microorganisms enables efficient metabolism of 3-picoline. Mutants with enhanced activity profiles are developed through gene-level modifications, ensuring superior catalytic efficiency and stability. Synthetic biology techniques generate tailored coding sequences for optimum expression. Synthetic constructs embedded with engineered genes, ribosomal binding sites, and spacers are co-expressed within one cellular unit. A one-pot reaction system utilizes versatile plasmid vectors like pET28a(+) for efficient co-expression, advancing the host microorganism matrix. The invention integrates immobilized whole-cell catalysts, addressing catalyst reusability, stability, and industrial scalability. Enhanced cell permeability and oxygen incorporation improve reaction efficiency and substrate accessibility, offering a scalable, cost-effective solution for industrial bioconversion processes.

The instant application contains a Sequence Listing which has been submitted electronically in XML file format and is hereby incorporated by reference in its entirety. Said XML copy, created on Sep. 24, 2024, is named Seqlisting.xml and is 53000 bytes in size.

### FIELD OF THE INVENTION

The present invention pertains generally to the fields of molecular biology, synthetic biology, microbiology, biochemistry and biocatalysis & to the application of computational biology in these areas. This invention involves the integration of various biological, biochemical, and computational methodologies to engineer enzymes and microbial strains, facilitating the optimized biocatalytic conversion of specific substrates. The domains of molecular and synthetic biology are central to understanding and manipulating genetic and biochemical pathways, enabling the design and synthesis of novel biological systems and entities for enhanced biocatalytic processes. Microbiology plays a critical role in utilizing microorganisms, such as Escherichia coli, as hosts for the engineered enzymes, while computational biology is pivotal for conducting structural studies and in silico modeling to predict and analyse the structural conformation and functional dynamics of the enzymes involved.

### BACKGROUND OF THE INVENTION

Nicotinic acid (NA), commonly known as niacin, is a B-vitamin of great significance. Fundamental to various physiological processes, it drives food conversion into energy, facilitates DNA repair, and aids in synthesizing certain stress-related hormones in the adrenal glands. Numerous studies, including CN Patent publication No. 106377609 by Zhang, T., have highlighted its therapeutic uses in maintaining cholesterol balance and potentially averting cardiovascular ailments. A majority of the industrially produced nicotinic acid, amounting to over 60%, finds its way into animal feed-benefiting poultry, fish and other domestic animals. Biologically, nicotinic acid serves as a precursor to nicotinamide, which ultimately paves the way for the synthesis of the crucial cofactor NAD (P) (Chuck R., 2000), the significance of which is underscored by their role in vital cellular metabolic activities, especially oxidoreduction reactions steered by enzymes. In addition to its pharmaceutical and nutraceutical utilities, nicotinic acid is enlisted by industries such as electroplating, where it acts as an anti-corrosive agent for steel (Lui X. et. al., 2020). In terms of economic scale, the global nicotinic acid market reached a staggering $614M in 2019. Over 90% of this is synthesized from starting materials such as 3-picoline otherwise known as 3-methylpyridine, or 5-ethyl-2-methylpyridine via an oxidation process (Lisicki, D. et. al., 2022).

3-picoline stands out as an optimal starting substrate for various transformative processes, largely due to its economical abundance and low cost. Known to be a byproduct of numerous industrial processes, it can be sourced in ample quantities. The chemical structure of 3-picoline lends itself to selective oxidation of the methyl group, resulting in a carboxylic acid with minimal byproducts. The compound's genesis can be traced to the gas-phase amalgamation of acetaldehyde, formaldehyde, and ammonia, which yields a mix of 3-picoline and pyridine. Given the affordability and ample availability of 3-picoline, biocatalytic conversions are not only scientifically exciting but also commercially profitable (Chuck R., 2000).

The Xylene Monooxygenase system (XMO) are enzymes from the xylene metabolizing pathway and are known for their ability to catalyze the oxidation of aromatic hydrocarbons. Specifically, it can oxidize the methyl groups of aromatic compounds like xylene, introducing a hydroxyl group. The XMO system consists chiefly of the xylene monooxygenase enzyme and two more dehydrogenase enzymes encoded by the XylM, XylB, and XylC genes, respectively. The monooxygenase enzyme is replenished by the activity of a ferredoxin domain containing enzyme which acts as the electron transfer component, encoded by the XylA gene. Post hydroxylation by the monooxygenase, the associated dehydrogenases, benzyl alcohol dehydrogenase (XylB) and the Benzaldehyde dehydrogenase (XylC), oxidize the aromatic alcohol into the aldehyde and subsequently the carboxylic acid, respectively. Numerous studies, including Gu et al., 2020; Luo, Z. W., et. al., 2017; Kiener, A. 1992; and He, S. et. al., 2023, have illuminated various applications of XMO, ranging from producing 5-methylpyrazine-2-carboxylic acid in E. coli to biotransforming p-xylene into terephthalic acid. In addition to these applications, the structural intricacies of XMO, with its dual subunits and a unique diiron core in the active site, makes it an enzyme of immense interest. XMOs show widespread activity due to their ability to oxidise and hydroxylate aromatic or heterocyclic aromatic molecules specifically at the terminal carbons. Some applications that will substantiate the use of XMO for the biocatalytic synthesis of NA from 3-picoline include,

High-Yield Production of 5-Methylpyrazine-2-Carboxylic Acid in E. coli:

An Escherichia coli strain is engineered to serve as a whole-cell biocatalyst by incorporating plasmid-mediated expression of XMO, benzyl alcohol dehydrogenase (BADH), and benzaldehyde dehydrogenase (BZDH) sourced from Pseudomonas putida ATCC 33015. The plasmid-free approach ensures a streamlined metabolic process, reducing cellular stress and enhancing overall yield (Gu et al., 2020)

Biotransformation of p-Xylene to Terephthalic Acid:

Another landmark study demonstrates the capability of engineered E. coli to transform p-xylene into terephthalic acid, a key compound in the polymer industry. Here, XMO plays a fundamental role in the oxidation steps, emphasizing its importance in industrial biotransformation processes (Luo, Z. W., et. al., 2017).

### Enzymatic Oxidation of Methyl Groups on Aromatic Heterocycles:

XMO's versatility is showcased in the enzymatic oxidation of various aromatic heterocycles. This approach introduces a method for preparing heteroaromatic carboxylic acids, compounds of significant interest in organic chemistry and drug design (Kiener, A. 1992).

Microbial Production of Cis, Cis-Muconic Acid from Aromatic Compounds in Engineered Pseudomonas:

This application of xylene monooxygenase pertains to environmental remediation. The ability of Xylene monooxygenases to degrade and metabolize aromatic compounds such as Benzene, Toluene, Ethylbenzene and Xylene are employed as a means for reducing the impact of monocyclic aromatic hydrocarbon pollutants (He, S. et. al., 2023).

Xylene refers to any of the three isomeric dimethyl derivatives of benzene (ortho-, meta-, and para-xylene). Among them, m-xylene is the most commonly occurring isomer. Given its aromatic structure, xylene is a natural substrate for XMO. While XMO has primarily evolved to act on substrates like xylene, its capability can extend to other similar aromatic and heteroaromatic compounds, including 3-picoline. However, the efficiency and specificity of this action can vary. When 3-picoline is subjected to XMO, there's potential for the enzyme to oxidize the methyl group, much like it does with xylene.

Both xylene and 3-picoline (3-methylpyridine) are organic aromatic compounds, and their structural similarities stem from their aromatic nature combined with the presence of a methyl group substitution. The core structure of xylene is a benzene ring, a six-carbon ring with alternating double bonds, making it aromatic. 3-picoline has a pyridine ring as its core structure. A pyridine ring is a six-membered ring, similar to benzene, but one of the carbons is replaced by a nitrogen atom. The “3-methyl” prefix indicates a methyl group substitution at the third carbon position of the pyridine ring.

Both xylene and 3-picoline possess aromatic rings which give them stability and specific chemical reactivity patterns typical of aromatic compounds. Both compounds have a methyl group (—CH₃) attached to their aromatic ring. This methyl group imparts certain chemical properties to the molecules, especially in reactions that target this functional group. Both molecules are relatively small and share a similar overall geometry due to the six-membered ring. However, it's important to note their difference, the presence of the nitrogen atom in 3-picoline. This heteroatom introduces a unique set of chemical properties when compared to the fully carbon-based benzene ring of xylene. The nitrogen in the pyridine ring of 3-picoline is electron-withdrawing, which influences the reactivity of the ring in certain chemical reactions.

Therefore, utilizing monooxygenases for converting 3-picoline to nicotinic acid presents its own set of challenges. Specifically, its affinity for 3-picoline may be subpar, as the enzyme has evolved to favour aromatic substrates such as xylene and toluene. For instance, XMO derived from P. putida showcases only a 50% conversion efficiency on 3-methylpyridine in contrast to a near-complete conversion on xylene (Kiener, A., 1992). Challenges further cascade in the form of substrate inhibition, product toxicity, and unanticipated side reactions (Kardashliev, T., et. al., 2022).

Given the profound economic and biological importance of nicotinic acid, and the untapped potential of 3-picoline as a starting material, there's a pressing need to navigate the complexities of XMO and harness its capabilities for a transformative synthesis. The present invention addresses this need, aiming to merge synthetic biology, metabolic engineering, and enzyme optimization to utilise monooxygenases for the synthesis of nicotinic acid from 3-picoline (Jumper J., et al., 2021), (Ogawa, Y., et. al., 2023).

### PRIOR ART

Austin, Rachel N., et al. “Xylene Monooxygenase, a Membrane-spanning Nonheme Diiron Enzyme That Hydroxylates Hydrocarbons via a Substrate Radical Intermediate.” Journal of Biological Inorganic Chemistry, vol. 8, no. 7, Springer Science+Business Media, June 2003, pp. 733-40. https://doi.org/10.1007/s00775-003-0466-3.
Chuck, Roderick. “A Catalytic Green Process for the Production of Niacin.” Chimia, vol. 54, no. 9, Swiss Chemical Society, September 2000, p. 508. https://doi.org/10.2533/chimia.2000.508.
Gu, Liuyan, et al. “High-yield and Plasmid-free Biocatalytic Production of 5methylpyrazine-2-carboxylic Acid by Combinatorial Genetic Elements Engineering and Genome Engineering of Escherichia coli.” Enzyme and Microbial Technology, vol. 134, Elsevier BV, March 2020, p. 109488.
   https://doi.org/10.1016/j.enzmictec.2019.109488.
He, Siyang, et al. “Microbial Production of Cis, Cis-muconic Acid From Aromatic Compounds in Engineered Pseudomonas.” Synthetic and Systems Biotechnology, vol. 8, no. 3, Elsevier BV, September 2023, pp. 536-45. https://doi.org/10.1016/j.synbio.2023.08.001.
Jumper, John, et al. “Highly Accurate Protein Structure Prediction With AlphaFold.” Nature, vol. 596, no. 7873, Nature Portfolio, July 2021, pp. 583-89. https://doi.org/10.1038/s41586-021-03819-2.
Kardashliev, Tsvetan, et al. “Efficient Synthesis of 2,6-bis(Hydroxymethyl)Pyridine Using Whole-cell Biocatalysis.” Green Chemistry, vol. 24, no. 9, Royal Society of Chemistry, January 2022, pp. 3651-54. https://doi.org/10.1039/d2gc00333c.
Kiener, A. “Enzymatic Oxidation of Methyl Groups on Aromatic Heterocycles: A Versatile Method for the Preparation of Heteroaromatic Carboxylic Acids.” Angewandte Chemie, vol. 31, no. 6, Wiley, June 1992, pp. 774-75. https://doi.org/10.1002/anie.199207741.
Lin, Bingcheng, et al. “Enhanced Production of N-acetyl-d-neuraminic Acid by Multi-approach Whole-cell Biocatalyst.” Applied Microbiology and Biotechnology, vol. 97, no. 11, Springer Science+Business Media, February 2013, pp. 4775-84. https://doi.org/10.1007/s00253-013-4754-8.
Lin, Bingcheng, and Yong Tao. “Whole-cell Biocatalysts by Design.” Microbial Cell Factories, vol. 16, no. 1, Springer Science+Business Media, June 2017, https://doi.org/10.1186/s12934-017-0724-7.
Lisicki, Dawid, et al. “Methods to Produce Nicotinic Acid With Potential Industrial Applications.” Materials, vol. 15, no. 3, Multidisciplinary Digital Publishing Institute, January 2022, p. 765. https://doi.org/10.3390/ma15030765.
Liu, Xia, et al. “Nicotinic Acid Derivatives as Corrosion Inhibitors for Mild Steel in Hydrochloric Acid Solutions: An Experimental and Computational Chemistry Study.” Journal of Adhesion Science and Technology, vol. 35, no. 1, Brill, July 2020, pp. 63-80. https://doi.org/10.1080/01694243.2020.1787934.
Luo, Zhidan, and Sang Yup Lee. “Biotransformation of P-xylene Into Terephthalic Acid by Engineered Escherichia coli.” Nature Communications, vol. 8, no. 1, Nature Portfolio, May 2017. DOI: 10.1038/ncomms15689
Ogawa, Yuki, et al. “Engineering the Substrate Specificity of Toluene Degrading Enzyme XylM Using Biosensor XylS and Machine Learning.” ACS Synthetic Biology, vol. 12, no. 2, American Chemical Society, February 2023, pp. 572-82. https://doi.org/10.1021/acssynbio.2c00577.
Ramaswamy, S., et al. “Structures of Horse Liver Alcohol Dehydrogenase Complexed With NAD+ and Substituted Benzyl Alcohols.” Biochemistry, vol. 33, no. 17, American Chemical Society, May 1994, pp. 5230-37. https://doi.org/10.1021/bi00183a028.
Ricklefs, Esther, et al. “Three-steps in One-pot: Whole-cell Biocatalytic Synthesis of Enantiopure (+)- and (−)-pinoresinol via Kinetic Resolution.” Microbial Cell Factories, vol. 15, no. 1, Springer Science+Business Media, May 2016, https://doi.org/10.1186/s12934-016-0472-0.
Rosano, Germán L., and Eduardo A. Ceccarelli. “Recombinant Protein Expression in Escherichia Coli: Advances and Challenges.” Frontiers in Microbiology, vol. 5, Frontiers Media, April 2014 https://doi.org/10.3389/fmicb.2014.00172.
Salis, Howard M., et al. “Automated Design of Synthetic Ribosome Binding Sites to Control Protein Expression.” Nature Biotechnology, vol. 27, no. 10, Nature Portfolio, October 2009, pp. 946-50. https://doi.org/10.1038/nbt.1568.
Shaw, Jeffrey P., et al. “Kinetic Studies on Benzyl Alcohol Dehydrogenase Encoded by TOL Plasmid pWWO. a Member of the Zinc-containing Long Chain Alcohol Dehydrogenase Family.” Journal of Biological Chemistry, vol. 268, no. 15, American Society for Biochemistry and Molecular Biology, May 1993, pp. 10842-50. https://doi.org/10.1016/s0021-9258 (18) 82062-2.
Vaughan, Peter A., et al. “Conversion of 3-cyanopyridine to Nicotinic Acid by Nocardia Rhodochrous LL100-21.” Enzyme and Microbial Technology, vol. 11, no. 12, Elsevier BV, December 1989, pp. 815-23. https://doi.org/10.1016/0141-0229 (89) 90055-0.
Yeung, Catherine K., et al. “Physical, Kinetic and Spectrophotometric Studies of a NAD (P)-dependent Benzaldehyde Dehydrogenase From Pseudomonas Putida ATCC 12633.” Biochimica Et Biophysica Acta—Proteins and Proteomics, vol. 1784, no. 9, Elsevier BV, September 2008, pp. 1248-55. https://doi.org/10.1016/j.bbapap.2008.04.015.
Zahniser, M. P. D., et al. “Structure and Mechanism of Benzaldehyde Dehydrogenase From Pseudomonas Putida ATCC 12633, a Member of the Class 3 Aldehyde Dehydrogenase Superfamily.” Protein Engineering Design & Selection, vol. 30, no. 3, Oxford UP, March 2017, pp. 273-80. https://doi.org/10x.1093/protein/gzx015.

### OBJECTS OF THE INVENTION

The primary objective of this invention is a biosynthetic method to produce nicotinic acid from 3-picoline using microbial biotransformation. For the conversion of 3-picoline to nicotinic acid, two atoms of oxygen must be incorporated into the sp³ terminal methyl attached to the pyridine ring. An objective of the present invention is to determine enzymes of the monooxygenase class from different microbial organisms for the specific incorporation of the first oxygen into the sp³ terminal methyl group and additional reductases, and dehydrogenases, each responsible for a specific reaction to further oxidize the primary alcohol obtained from the first oxygenation to yield the final carboxylic acid product. Another objective of the present invention is to use multiple strategies such as use of single or multiple expression vectors or CRISPR-Cas9 based genome modification for cloning and expressing these genes in an established host such as E. coli, a model microorganism renowned for its versatile metabolic capabilities and well-characterized genetic landscape. This will ensure faster synthesis and expression of the isolated genes for efficient catalytic process. Another objective of the present invention is to provide precise engineered genes that show improvement in the respective biocatalytic activities over their native counterparts to optimize MO enzyme system performance achieving a conversion rate of 90% or higher in the transition from 3-picoline to nicotinic acid.

### SUMMARY OF THE INVENTION

The invention describes a methodology involving gene isolation, enzyme engineering, and synthesis optimization for efficient bioconversion of 3-picoline to nicotinic acid, using genes from selected organisms including Pseudomonas putida. Genes from the selected organisms are isolated for their unique metabolic assimilation capabilities, focusing on enabling the metabolic assimilation of aromatic substances as exclusive carbon sources. The aromatic metabolising enzymes ae engineered to metabolize 3-picoline the pyridine being structurally a heteroaromatic ring. The monooxygenase system which includes the monooxygenase enzyme, the electron transfer component of the monooxygenase, a benzyl alcohol dehydrogenase and a benzaldehyde dehydrogenase are singled out and further optimized to improve the catalytic efficiency of converting 3-picoline to nicotinic acid. The optimization of the MO system involves gaining mechanistical insights using QM/MM studies and structural insights, using a pLDDT-based Protein Optimization Protocol (P-POP) to generate mutants with enhanced activity profiles. Coding sequences are generated for optimum expression within the Escherichia coli or suitable microbial host environment using a one-pot reaction system, focusing on the four key genes vital for converting 3-picoline to nicotinic acid. Genes are co-expressed in one cellular unit, with synthetic constructs embedded with ribosomal binding sites and spacers for strengthened expression efficiency. Various vectors, with pET28a(+) vector as a primary choice, were used to create diverse gene constructs, each ensuring efficient expression. The importance of restriction enzyme sites is highlighted for future cloning, with unique site considerations being addressed for optimal cloning pursuits. Strategic placement of genes into these designated sites was vital to avoid any obstacles to expression levels. Additionally, diverse combinations are meticulously formulated to address concerns related to protein folding, thereby ensuring the successful production of desired variants. Immobilization techniques were implemented to overcome challenges related to the reusability and stability of whole-cell catalysts in industrial applications. This involves using the CaCl₂-Sodium alginate method for immobilizing recombinant host cells. Direct oxygen incorporation was integrated to amplify the biocatalytic efficiency of oxygen-dependent reactions in larger scales. The invention emphasizes enhancing cell permeability using non-ionic detergents like Tween 80 and TritonX to ensure efficient enzymatic reactions and substrate permeation into the cytosol.

### BRIEF DESCRIPTION OF FIGURES

FIG. 1. Reaction scheme to depict the synthesis of nicotinic acid from 3-picoline. 3-picoline is first hydroxylated in the terminal methyl group by the non-heme diiron catalytic site of the monooxygenase enzyme to form Pyridin-3-ylmethanol. Monooxygenase is replenished by an electron transfer protein. Pyridin-3ylmethanol then undergoes oxidation in the presence of the zinc-based Benzyl alcohol dehydrogenase enzyme to derive 3-pyridinecarboxaldehyde which is subsequently oxidized to nicotinic acid by the activity of enzyme Benzaldehyde dehydrogenase. The genes that code for the enzymes monooxygenase, the electron transfer protein, benzyl-alcohol dehydrogenase and benzaldehyde dehydrogenase are “M”, “A”, “B” and “C”, respectively.

FIG. 2. The membrane bound monooxygenase and the coupled electron transfer component. The monooxygenase houses the monooxygenase domain with the non-heme diiron catalytic center for hydroxylation of the terminal carbon attached to an aromatic ring. The electron transfer component is a reductase enzyme, housing the FADH domain and the ferredoxin domain that are involved in the replenishment of the electrons required by the monooxygenase domain.

FIG. 3. Schematic representation of the tetrameric complex of the benzyl alcohol dehydrogenase. Each monomeric unit binds two Zn²⁺ ions (spheres) and one NAD⁺ cofactor (sticks). The two Zn²⁺ ions are differentiated based on the function. One of the Zn²⁺ ions is characterized by the Zn²⁺ ion binding in a tetrahedral conformation with two cysteine residues and one histidine residue. This Zn²⁺ ion is necessary of the catalytic activity as it binds the oxygen atom of the substrate. The second Zn²⁺ ion is involved in structural stability, characterized by the Zn²⁺ ions binding in the tetrahedral conformation with four conserved cysteine residues. The benzyl alcohol dehydrogenase catalyses the reversible oxidoreduction of aromatic alcohol to aromatic aldehyde in the conversion of 3-picoline to Nicotinic acid.

FIG. 4. Schematic representation of the benzaldehyde dehydrogenase tetrameric complex each chain coloured differently. Each monomeric chain houses an NAD+ cofactor (sticks) which is complexed in the Rossman fold containing NAD (P) binding domain, designated by the “5-point star” symbol. The benzaldehyde dehydrogenase oxidizes the aldehyde to derive the aromatic carboxylic acid in the conversion of 3-picoline to Nicotinic acid.

FIG. 5. Quantum chemical study of the terminal hydroxylation reaction catalysed by the non-heme diiron catalytic center of the monooxygenase domain. The study aims to delineate the activation energies required for the reaction to proceed from the substrate (ground state, GS) to the product (product state, PS) through the formation of transition states and intermediate states (TS1, INT1, TS2). The energy gap between the GS and the first transition state, TS1 (9.5 kcal. Mol⁻¹) was proposed to be the rate-limiting step as it required relatively the highest activation energy. Comparatively, the energy required to form the second transition state (TS2) from the intermediate state (INT1) was determined to be 4.5 kcal·mol⁻¹. Despite the higher energy state of TS2 (10.4 kcal·mol⁻¹), it was not determined as the rate-limiting step as the energy gap between the INT1 and TS2 states was not higher than the energy gap between the GS and TS2 states. The product formed was at a state lower than the ground state (−2.2 kcal·mol⁻¹). The transition and intermediate states play a crucial role in determining the enzyme active site environment required for the reaction to proceed and therefore can provide insightful knowledge for optimization and engineering of the enzyme.

FIG. 6. Schematic representation or overview of the process of the pLDDT-based protein optimization protocol (P-POP). P-POP method is a protein engineering method to derive optimized enzyme variants for a specific functional requirement. A 3D structure of the enzyme was studied, and hotspots were derived from Rational-based approach and particular residues with lower pLDDT scores. An evolutionary analysis using a phylogeny-based approach was used to determine the substitution mutations for the hotspots and these substitutions were validated based on a pLDDT-scoring method, wherein an improvement in the pLDDT-score post mutation when compared to the pLDDT-score of the same position in the wild-type protein was desired. Top scoring variants were validated in vitro, and the results were used to further refine the hotspot selection, and substitution protocols. The final variants are selected through parameter optimization of the screened variants.

FIG. 7. Schematic representation of the determination of hotspots for the engineering study. Residues are coloured as a gradient based on their pLDDT scores (Red to green, with scores below 90.0 being treated as red). The conserved catalytic residues are coloured blue and the residues in the immediate vicinity of the catalytic zinc (Blue sphere) and NAD (Magenta sticks) cofactor are coloured in grey. Two residues with lower pLDDT scores P₁ and P₂ were considered as hotspots. For these two positions, evolutionary analysis and pLDDT score validation was used to determine best probable substitutions. Evolutionary analysis was used to determine the probability (Pxᵢ) of each amino acid (x) to occur at position Pᵢ as a function of the frequency of amino acid x occurring at position i (fₓᵢ) and the total number of sequences studied (N). The heatmap on the right indicates the Pₓ₁ and Pₓ₂ scores, and each cell is coloured based the pLDDT score for the respective amino acid substitutions at positions P₁ and P₂, respectively. The most probable substitution for each position is highlighted with the blue outlines. ‘*’ in the heatmap indicates the wild-type residue at the respective positions

FIG. 8. Schematic representation of the gene constructs described in this embodiment for the conversion of 3-picoline to nicotinic acid. Underline represents a schematic diagram of gene vector in the linearized form. The vectors were chosen from a selection of bacterial expression plasmids such as pET28a(+), pRSFDuet-1, pCDFDuet-1 and pETDuet-1, labelled appropriately at the end of each construct. Semi-circle represents ribosomal binding site (RBS) and optimized nucleotide spacer. Hourglass figures represent various restriction sites. Block arrow diagrams represent genes. Gene C, M, A and B, correspond to the genes encoding Benzaldehyde dehydrogenase (C), Xylene monooxygenase (M), electron transfer component of xylene monooxygenase (A) and benzyl-alcohol dehydrogenase (B). White block diagrams represent genes isolated from Pseudomonas putida pWWO and shaded block diagram represent genes isolated from Pseudomonas putida F1. Curved arrow represents the vector's innate promotor sequence. Crossed out circle represents the stop codons, small “T” diagram represents the vector's terminator codon. Dotted box indicates multiple cloning sites in the same vector. Constructs designed by considering the gene fragments from the genome of P. putida pWWO (Construct C, Construct D and Construct F) and P. putida F1 (Construct E, and Construct G) majorly consist of entire genome components and are represented as containing components within the block diagram. Additionally, they contain non-coding regions innate to the genome components as represented by the elongated hexagons.

FIG. 9. Schematic representation of the experimental setup of the external oxygen supply experiment. The setup consists of a reaction vessel, containing the reaction mixture made up of the substrate, the media components and whole cell biocatalysts. The reaction vessel comes with a septum-stoper, with a single opening. This opening is connected to an external oxygen supply using a valve and compressor setup. External oxygen is supplied as the reaction progresses by opening the valve to the desired extent.

FIG. 10. 10% SDS Polyacrylamide gel electrophoresis of constructs with wild genes. Lane 1-10 show optimised expression of whole-cell biocatalysis E. coli BL21 (DE3) harbouring construct D in Lane 1, Construct C in Lane 2, Construct C+B1 in Lane 3 and a duplicate of the same in Lane 4, Constructs D+B1 in Lane 5, construct C with two intermittent addition of IPTG to the culture medium in Lane 6 & a duplicate of the same in Lane 7, Constructs E+B1 in Lane 8 followed by its duplicate in Lane 9 & its triplicate in Lane 10. Arrows indicate the induction bands appeared at 41.5 and 38.45 kDa.

FIG. 11. 10% SDS Polyacrylamide gel electrophoresis of Construct B. Lane Un shows whole-cell biocatalysis E. coli BL21 (DE3) harbouring uninduced variant; Lane 2 shows whole cell of E. coli BL21 (DE3) harbouring constructs B1+B2+B3 mutant; Lane 3 shows whole cell of E. coli BL21 (DE3) harbouring Constructs B1+B2+B3; Lane 4 shows whole cell of E. coli BL21 (DE3) harbouring Constructs B1+B3 mutant; Lane 5 shows whole cell of E. coli BL21 (DE3) harbouring Constructs B1+B3; Lane 6 shows whole cell of E. coli BL21 (DE3) harbouring Construct B3 mutant. Arrows indicate the induction bands appeared at 41.5, 38.45 kDa.

FIG. 12. 10% SDS Polyacrylamide gel electrophoresis of Construct A. Gel image shows whole-cell biocatalysis E. coli BL21 (DE3) harbouring Construct A with engineered genes generated by SDM. Lane M shows medium range Molecular weight marker; Lane 1-8 Expression of whole cell biocatalysis E. coli BL21 (DE3), Mutants 1, 2, 3, 4, 5, 8, 9, 10 respectively. Arrows indicate the induction bands appeared at 41.5, 38.45 kDa.

FIG. 13. 0.8% Agarose gel electrophoresis of PCR amplified Construct-A containing engineered genes generated by SDM. A) Lane M shows 1 kb molecular weight marker; Lane 1-5 show expression of construct A mutants 1-4 and construct A mutant 41 respectively. B) Lane 1-6 show expression of construct A mutants 5, 8, 9, 10, 12 and 13. C) Lane 1 and 2 show expression of construct A mutants 14 and 15.

The patent or application file contains at least one drawing executed in color. Copies of this patent or patent application publication with color drawings will be provided by the Office upon request and payment of the necessary fee.

### DEFINITION/EXPLANATIONS TO KEYWORDS

### Reference Protein

“Reference Protein” in this context refers to any one of the proteins encoded by the “M”, “A”, “B” or “C” genes. Specifically, it refers to the protein studied for engineering and optimization of desired functions.

### Genes “M”, “A”, “B”, and “C”

Genes “M”, “A”, “B”, and “C” refer to the genes that encode the monooxygenase, the electron transfer component of the monooxygenase, the benzyl alcohol dehydrogenase and the benzaldehyde dehydrogenase enzymes, respectively.

### Rational Based Design

“Rational based design” in this context refers to the method of engineering the enzyme by choosing hotspots and substitutions from a visual understanding of the 3D-protein structure. The hotspots and their respective substitutions are proposed based on the rational understanding that the mutation will bring about the desired functionality.

### Parameter Optimisation

“Parameter optimisation” in this context refers to the optimization of the reaction conditions such as pH, temperature, substrate loading, enzyme loading, enzyme-substrate loading, co-substrate loading, co-factor loading, enzyme cofactor loading, solvent concentration, cell permeability, external oxygen supply, dry cell weight of the whole cell biocatalysis etc.

### AlphaFold

AlphaFold is an AI tool developed by DeepMind which can predict the 3D structure of a given protein from a sequence input using well trained Artificial intelligence models to derive structures at atomistic levels of accuracy. AlphaFold's neural network-based approach models the entire protein chain in the context of its surrounding environment. The network predicts the angles between amino acid residues and their distances from each other, which ultimately defines the 3D structure. The AlphaFold method was used to determine the 3D-protein structures used in this study.

pLDDT

pLDDT (Predicted LDDT): LDDT stands for Local Distance Difference Test. It is a metric used to assess the quality of predicted protein structures by comparing the predicted local distances between amino acid residues to the actual local distances found in experimentally determined structures. pLDDT, as implemented in the AlphaFold system, is the predicted version of this metric. pLDDT scores are given for each residue in the protein, which means it provides a local assessment of prediction accuracy. This allows researchers to identify regions of the predicted structure that are more likely to be accurate, and those which might be less reliable. pLDDT scores range from 0 to 100, with higher scores indicating greater confidence in the predicted structural accuracy for that residue. Structures with scores above 90 are generally considered to be of very high accuracy and comparable to experimental data. In practical applications, pLDDT offers scientists a gauge on the reliability of the predicted structure. While a high overall pLDDT score indicates that the predicted structure is likely accurate throughout, a protein with variable scores across its length might have regions of high certainty and others that are more tentative. In conclusion, pLDDT is an integral part of AlphaFold's protein structure prediction system, serving as a measure of confidence and aiding in the interpretation and validation of the predicted structures. Additionally, pLDDT is used as a scoring function in the enzyme optimization protocol mentioned in this embodiment.

### Evolutionary Analysis

“Evolutionary analysis” in this context refers to a method of determining the substitutions for hotspots determined by the pLDDT based protein optimization protocol. Substitutions were determined through phylogenetic analysis of homologs and related sequences. Multiple sequence alignment was performed on all the sequences involved in the study. The sequence of interest was used as the parent sequence. Probability of a particular residue to occur in a chosen hotspot was determined as a function of the frequency as given by the equation: Pxᵢ=(f/n)*100, where Pxᵢ is the probability of ‘x’ amino acid corresponding to ‘i’^{th} position of the sequence of interest in the multiple sequence alignment without taking gaps into consideration, f is the frequency or number of times the ‘x’ amino has occurred in the i^{th} position and n is the total number of amino acids corresponding to i^{th} position of the sequence alignment. Higher the evolutionary probability, greater the chances of selecting the amino acid substitution of ‘x’ at the i^{th} position.

### MO System

“MO System” in this context refers to the collective term given to four enzymes encoded by genes for the specific conversion of 3-picoline to nicotinic acid. Specifically, we refer to the monooxygenase enzyme encoded by the “M” gene, the electron transfer component of the monooxygenase encoded by the “A” gene, a benzyl alcohol dehydrogenase encoded by the “B” gene, and a benzaldehyde dehydrogenase encoded by the “C” gene.

### DETAILED DESCRIPTION OF THE INVENTION

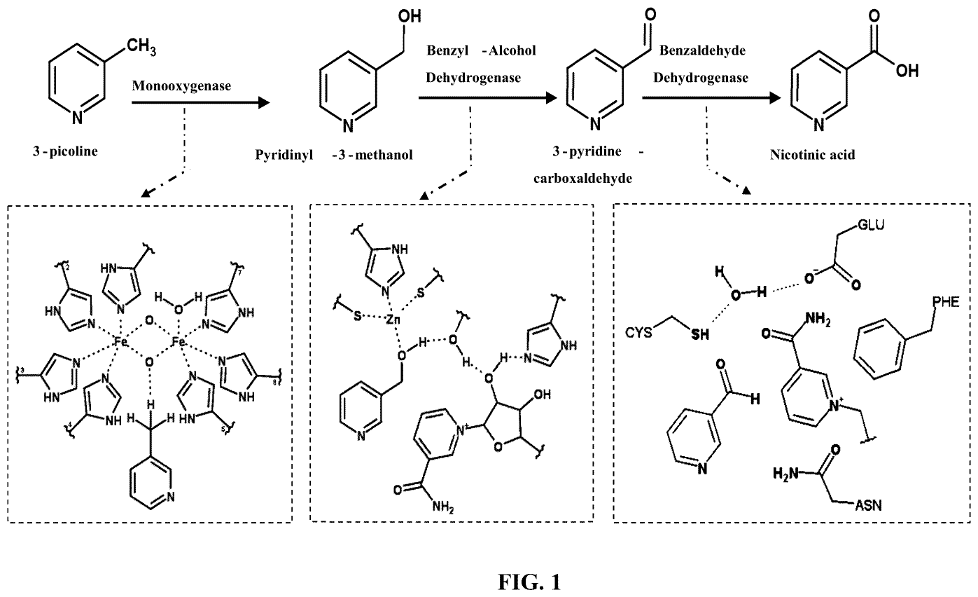

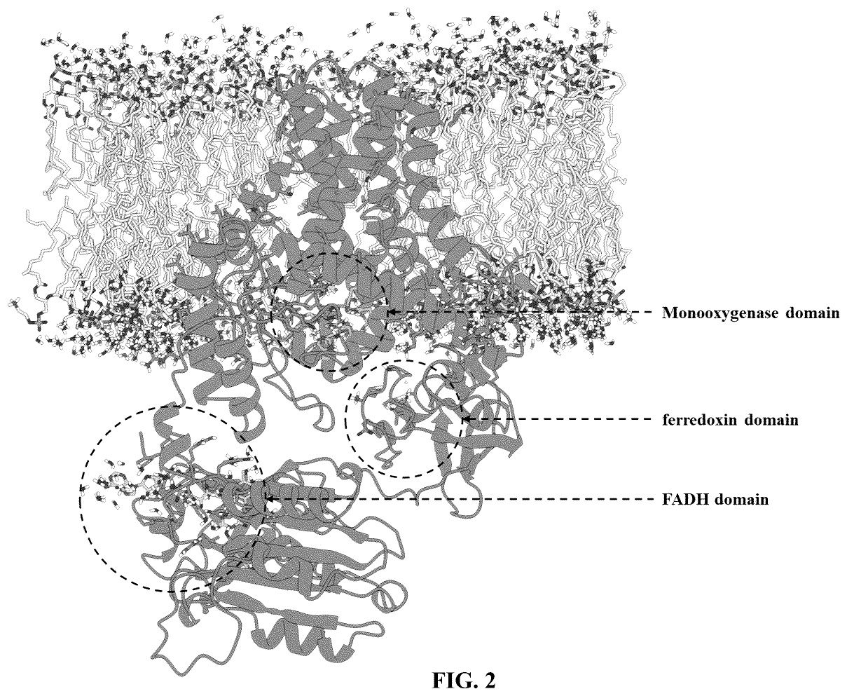

The present invention is a methodology to synthesise Nicotinic acid (NA) from 3-picoline by a one pot reaction process as shown in FIG. 1. The conversion requires four proteins, the monooxygenase enzyme encoded by the “M” gene, Monooxygenase replenishing, electron transfer protein encoded by the “A” gene, Benzyl-alcohol dehydrogenase encoded by the “B” gene and the Benzaldehyde dehydrogenase encoded by “C” gene. The method makes use of synthetic gene constructs comprising the above-mentioned genes to do the conversion in single pot. The synthetic gene constructs are transformed into recombinant E. coli and made to over-express the genes. Monooxygenase is responsible for hydroxylating the methyl group in the aromatic or heteroaromatic molecule. The alcohol formed is oxidized by benzyl alcohol dehydrogenase to yield an aldehyde, and subsequently oxidized by benzaldehyde dehydrogenase to yield the carboxylic acid. In some cases, monooxygenases can convert aromatic or heteroaromatic alcohol to aldehyde, without the need for benzyl alcohol dehydrogenase, in which case, the reaction conditions would call for a recombinant organism expressing only three genes, i.e., “M”, “A” and “C”, and omitting “B”. The synthetic gene constructs described in this invention are constructed using plasmid vectors pET28a(+), pCDFDuet-1, PRSFduet-1, and PETDuet-1 expressing the genes encoding for the required enzymes either individually or in combination as needed.

The Monooxygenase System (MO system)

The Monooxygenase comprises of two heteromeric subunits, the monooxygenase enzyme and the electron transfer component reductase subunit. The Monooxygenase enzyme encoded by the “M” gene is responsible for the hydroxylase activity and comprises a non-heme diiron core in the active site (FIGS. 1 & 2). The two iron atoms in the IV oxidation state, are bound in the active site by a total of seven histidine side chains, two oxygen atoms and a water molecule in a trigonal-bipyramidal arrangement for each atom. The reaction proceeds through the proton abstraction from the substrate terminal carbon by the oxygen atom bound to the diiron core. The radical thus formed is attacked by the oxygen atom thereby transferring a hydroxyl group to the terminal carbon. The remaining oxygen in the diiron center is reduced by the associated reductase domain of the electron transfer protein. The electrons required for the hydroxylation activity is provided by the ferredoxin containing domain of the reductase subunit encoded by the “A” gene. The reductase subunit houses an FAD domain that oxidizes NADH while enabling the reduction of the other oxygen atom in the diiron catalytic center to a water molecule. The monooxygenase domain is majorly comprised of 7 helices rich in hydrophobic residues which enables the monooxygenase to integrate into the membrane (Austin R. N., et. al., 2003).

### Benzyl-Alcohol Dehydrogenase

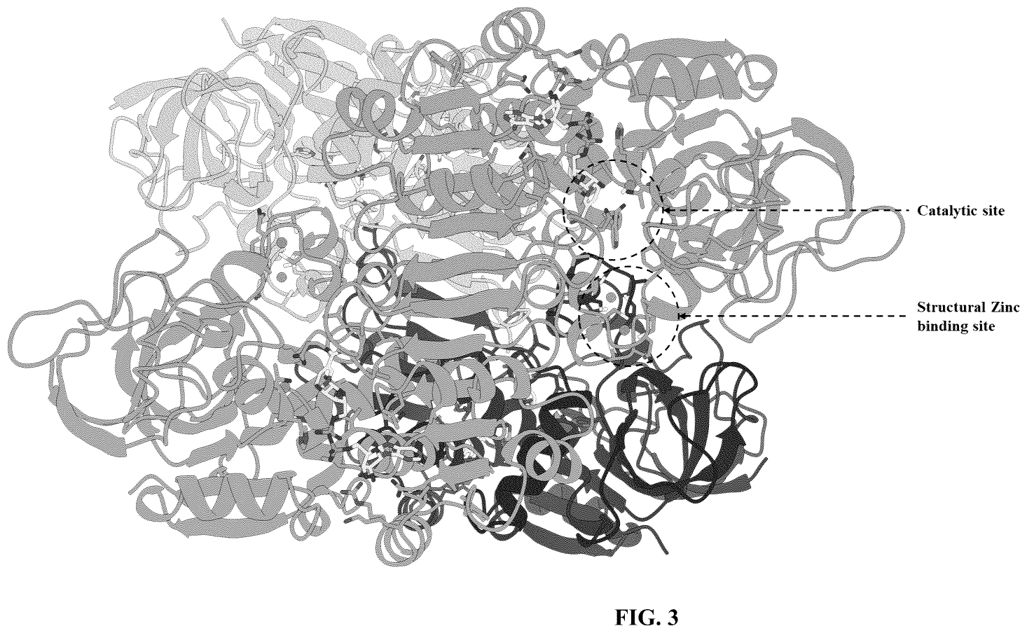

Benzyl alcohol dehydrogenase, encoded by the “B” gene, belongs to the class of Zinc-containing long chain alcohol dehydrogenases (Figure. 3). These enzymes catalyse the reversible oxidoreduction of benzyl alcohol to benzaldehyde with the use of an NAD cofactor [Shaw J, P., et al., 1993]. These enzymes belong to the same class of alcohol dehydrogenases as that of the horse liver alcohol dehydrogenase. The Zinc-based ADH is functionally characterized as a tetramer with each monomeric unit binding two Zn²⁺ ions (a catalytic and a structural). The structural Zn²⁺ ion is positioned by metallo-cysteine bonds provided by conserved cysteine residues. The catalytic Zn²⁺ ion is placed in a pocket adjacent to a hydrophobic cleft. This Zn²⁺ ion is stabilized in the active site by the side chains of two cysteine residues and the imidazole group of a histidine residue. In some cases, one of the two zinc binding cysteine residues is replaced with an aspartate group. The nicotine-amide moiety is positioned adjacent to the Zn²⁺ ion and the hydrophobic substrate binding cleft which is closed using a rigid body rotation of the different monomeric units post complex formation. The substrate binds in an orientation such that the oxygen atom of either a hydroxyl group or a carbonyl group faces the Zn²⁺ ion. The reaction proceeds through the transfer of the proton using a relay that involves a serine residue and the ribosyl moieties of the NAD cofactor. The reaction is then completed by the transfer or abstraction of hydride in the case of reduction or oxidation reactions, respectively (Ramaswamy, S. et al., 1994).

### Benzaldehyde Dehydrogenase

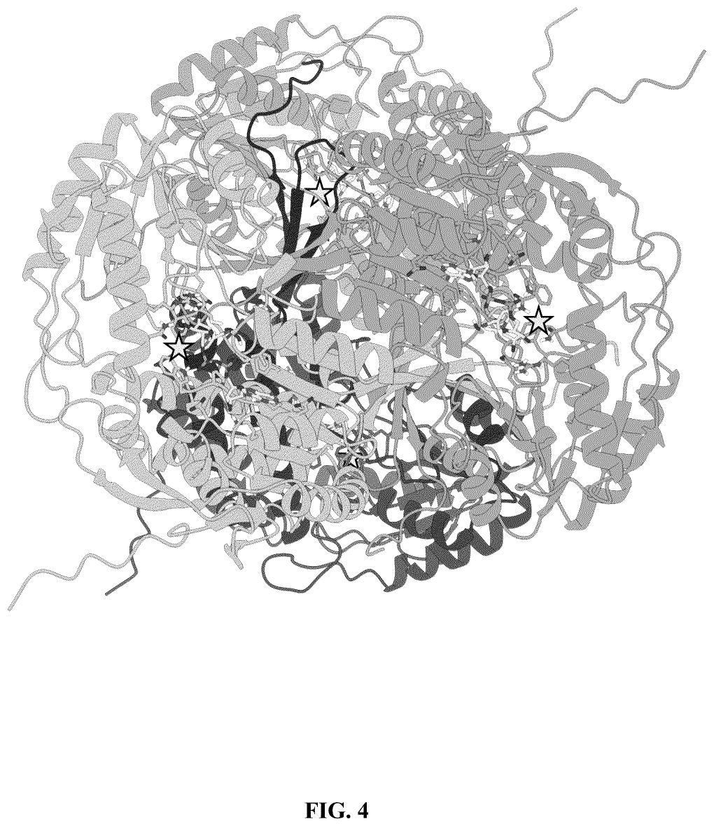

Benzaldehyde dehydrogenase, encoded by the “C” gene, occurs as a homodimer in solution, with each monomer containing a nucleotide cofactor binding domain and the catalytic binding domain (Figure. 4). The dimerization is such that the bridging domain between the two monomers forms part of the other monomer's substrate access channel. The nucleotide cofactor binding domain in this case, binds the nicotinamide adenine dinucleotide (phosphate) (NAD (P)) cofactor in a Rossmann fold motif. The catalytic residues include a cysteine and an aspartic acid with the nicotine-amide moiety of the NAD (P) cofactor positioned in the same cavity to facilitate hydride transfer (Zahniser, M. P. D., et. al., 2017). The reaction proceeds with the deprotonation of the active site cysteine by the nearby aspartate residue. The deprotonated thiol acts a strong base and attacks the carbonyl carbon of the aldehyde substrate forming a thiohemiacetal intermediate. The NAD (P) cofactor then abstracts a hydride from the intermediate thereby getting reduced to the NAD (P) H state and forming an acyl-enzyme intermediate which is hydrolysed in the presence of water to release the acid product (Yeung C. K., et. al., 2008)

In order to make the synthetic gene construct of the above described genes, the method involves the isolation, manipulation, and expression of genes sourced from Pseudomonas putida, Arthrobacter woluwensis, Acidovorax sp., Acinetobacter calcoaceticus, Burkholderia sp., Croceicoccus sp., Cupriavidus sp., Delftia sp., Devosia sp., Geodermatophilus sp., Jatrophihabitans sp., Kribella sp., Lacisediminimonas sp., Microbacterium sp., Mycolicibacterium sp., Nocardioides sp., Novosphingobium sp., Parapusillimonas sp., Planosporangium sp., Prauserella sp., Ramlibacter sp., Rhodococcus sp. into heterologous host expression systems such as Escherichia coli (E. coli), Saccharomyces cerevisiae, Pichia pastoris, Schizosaccharomyces pombe, Aspergillus niger, Trichoderma reesei, and Penicillium chrysogenum.

### Heterologous Expression of MO System

The expression of the MO system in non-native hosts, known as heterologous expression, is a strategy employed to study the enzyme in a controlled environment and to understand its catalytic potential for biotechnological applications. Using platforms like E. coli will help in producing large amounts of MO system, enabling detailed biochemical and structural studies, as well as scaled-up biotransformation. Advances in microbiology has led to versatile usage of E. coli in the expression of recombinant DNA due to the features of the organism such as fast growth kinetics, high cell densities, relative ease of transformation, and the ability to grow on simple or rich complex media derived from readily available and inexpensive components. Specifically, the commercially available E. coli BL21 (DE3) strain was used due to aspects such as reduced DNA methylation and degradation. This strain also contains the λDE3 prophage DNA incorporated into the protein which codes for the T7RNA polymerase (T7RNAP). The T7 promotor present in most expression vectors such as the pET-based plasmids require the expression of the highly active T7RNAP and are induced by the presence of Lactose or the non-hydrolysable analogue, isopropyl β-d-1-thiogalactopyranoside (IPTG). The IPTG induction results in an expression of the recombinant protein as 50% of the total cell protein. (Rosano, G. L., et al., 2014) Ribosomal binding sites (RBS) are independent of the promotor and can directly affect protein translation. By optimizing and choosing the correct RBS and spacer sequence between the promotor and the start codon of the recombinant gene, initiation of translation by the binding of 30S ribosomal subunit can be regulated, thereby directly affecting the rate of translation (Salis, H. et al., 2009)

### The Methodology Involves the Following Steps

### Gene Isolation and Sources:

Key to this procedure is the intricate isolation of genes situated within the Pseudomonas putida TOL plasmid. The significance of this plasmid arises from its capacity to produce genes that enable the metabolic assimilation of aromatic substances as exclusive carbon sources. To ensure the highest fidelity, gene sequences were diligently acquired from the National Centre for Biotechnology Information (NCBI) website and a gene web browser, pertaining directly to relevant genome sequences.

### Enzyme Engineering of MO to Generate Mutants

The enzymes involved in aromatic compound metabolism could be utilized to metabolize 3-picoline, given the structural nature of 3-picoline (3-picoline has a methyl group attached to an aromatic ring). However, there are some considerations and challenges as given below

Substrate Specificity: Enzymes typically have a degree of substrate specificity. Even though 3-picoline is structurally an aromatic molecule, the presence of the nitrogen atom in the pyridine ring of 3-picoline can influence how the molecule interacts with the enzyme. The enzyme's active site might not accommodate 3-picoline as effectively as aromatics, leading to reduced efficiency or altered enzyme activity.

Enzymatic Mechanism: The mechanism of oxidation by enzymes like monooxygenase (MO) on aromatics might differ slightly when it comes to 3-picoline. The reaction intermediate or transition states could be different given the different electronic properties of pyridine compared to benzene.

Metabolic Pathway Interactions: Even if the initial oxidation steps are successful, the downstream metabolic processing of the resulting intermediates might not be as straightforward. The cell's native enzymes might not readily convert the intermediates derived from 3-picoline as they would for other aromatic molecules.

Toxicity and Feedback Inhibition: The intermediates or products derived from 3-picoline metabolism might be inhibitory or toxic to the cell, leading to feedback inhibition or cellular stress.

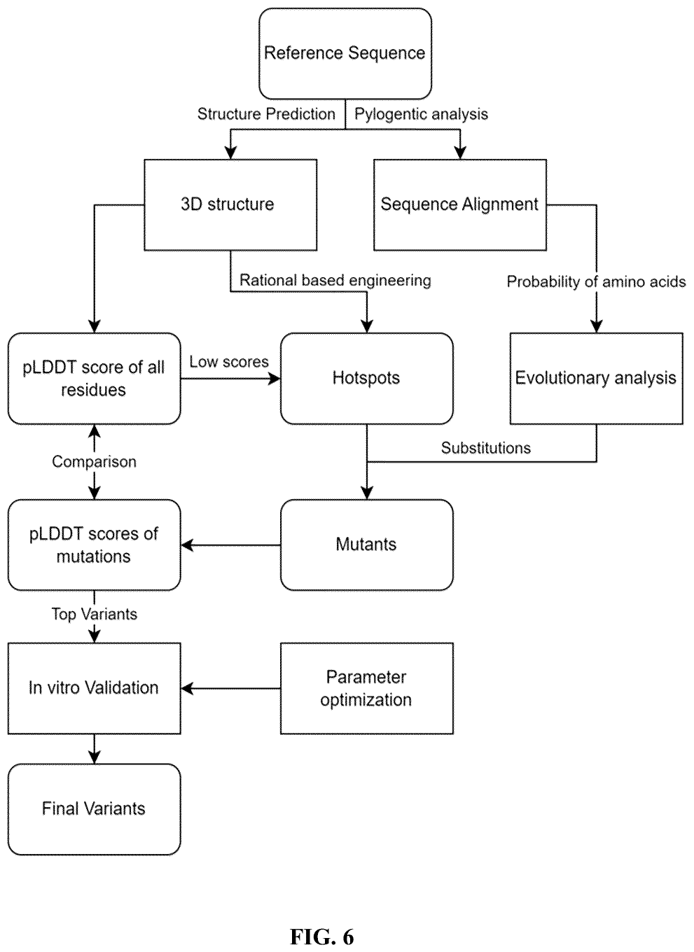

Tools such as the AlphaFold with its pLDDT scoring function with a machine learning approach to predict the structure of proteins with atomic level accuracy were used. For MO, this involves understanding how it interacts with substrates at the atomic level. These models predicted how modifications to the enzyme might influence its activity, guiding experimental efforts. By understanding the mechanistic details of MO, mutants with enhanced or altered activity profiles were rationally designed. The advancement of this catalyst arises from an understanding of the reaction mechanism or behaviour of the enzyme as is achieved by a series of steps shown in FIG. 6, primarily informed by in silico modelling using (a) QM/MM studies and from inhouse developed protocol used for enzyme engineering, (b) PLDDT-Based Protein Optimization Protocol (P-POP).

### QM/MM Simulations & Mutations:

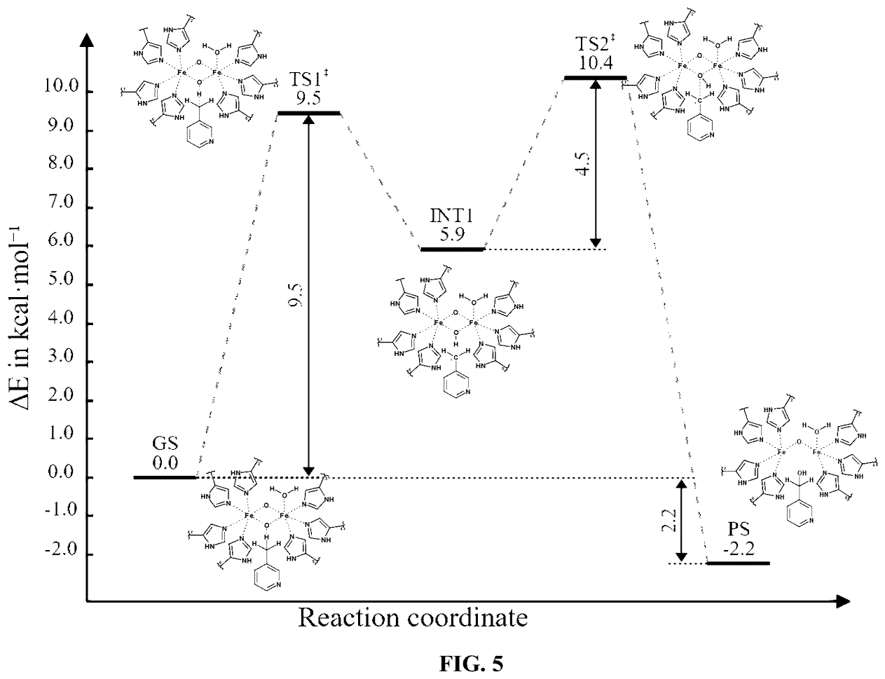

Mapping the Reaction Pathway: The entire reaction sequence was traced from the reactant, through intermediate states, to the product. Each of these states were energetically evaluated, for a clear picture of the potential barriers and favourable conditions; an example is shown in FIG. 5.

Intermediate States: The intermediate states represent transient molecular configurations that the reacting molecules assume during their conversion. These states are fleeting and often hard to detect experimentally, but they are of immense importance. By understanding their structure and stability, we can predict the rate and success of the reaction. For the MO catalytic system, these intermediate states were rigorously analysed to determine their role in achieving the desired product.

Efficiency and Selectivity of MO system: Through in silico modelling, we were able to understand how the MO system catalyst interacts with 3-Picoline and its intermediates. This interaction dictates the efficiency (how fast the reaction proceeds) and selectivity (how often the desired product is formed compared to undesired by-products).

Quantum Mechanics/Molecular Mechanics (QM/MM) simulations were employed to study the detailed electronic and structural properties of the system. These simulations provided atomic-level insights into how the MO catalytic system facilitates the conversion process. Armed with this knowledge of the role of the active site residues, mutations on the MO system were predicted. These mutations are essentially proposed changes to the molecular structure of the MO catalyst that could potentially enhance its performance.

### PLDDT Based Protein Optimization Protocol (P-POP)

The process of converting 3-Picoline to nicotinic acid using the MO catalytic system is complex and involves several intermediate steps. One of the primary tools used to understand and optimize this conversion is in silico modelling. This computational technique allows us to probe the molecular and atomic-level details of the reaction pathway, an understanding that is often hard or impossible to achieve through purely experimental means.

The present invention pertains to the field of protein engineering and optimization. Specifically, the invention describes a protocol that combines computational predictions using pLDDT scores from the AlphaFold system with practical protein engineering methodologies.

The present invention introduces the pLDDT-Based Protein Optimization Protocol (P-POP), designed to iteratively improve protein properties by leveraging pLDDT scores to guide in silico mutations and subsequent experimental validation.

### Detailed Description of the PLDDT-Based Protein Optimization Protocol (PPOP):

Objective Definition: Before starting the protocol, the property or function aimed to be optimized in the target protein, such as enzymatic activity, stability, or binding affinity is defined.

Preliminary Structure Prediction: The 3D structure of the wild-type protein is predicted using the AlphaFold system, and pLDDT scores for each residue are extracted to provide a baseline.

Hotspot selection: Hotspots are chosen based on low pLDDT score and the regions are chosen from the core (Active site) to the periphery of the enzyme. The fraying regions like the n-terminal and c-terminal are not chosen for hotspot selection.

Mutation Selection: Potential mutation substitutions are chosen based on the Evolutionary analysis.

Mutant Structure Prediction: For each proposed mutation, its structure is predicted in silico using AlphaFold. pLDDT scores for the mutated residue and its neighbours are derived.

Analyzing pLDDT Scores: pLDDT scores from the mutated protein are compared against the wild type. Significant drops in scores may indicate potential structural issues, while stable or increasing scores might suggest compatibility.

In Vitro Validation: Promising mutants, based on pLDDT score insights, are expressed or synthesized in vitro. Experimental validation is then conducted to confirm the desired activity or property.

Iterative Refinement: Results from the in vitro validation guide further refinement of mutation sites. Steps [0057-0062] are repeated as necessary to approach the desired optimization goal.

Final Validation: Upon identifying an optimized protein variant, it undergoes rigorous testing under various conditions to confirm its enhanced properties and practical applicability.

Documentation: All pLDDT score changes and their correlations with experimental results are systematically documented, aiding in refining future predictions.

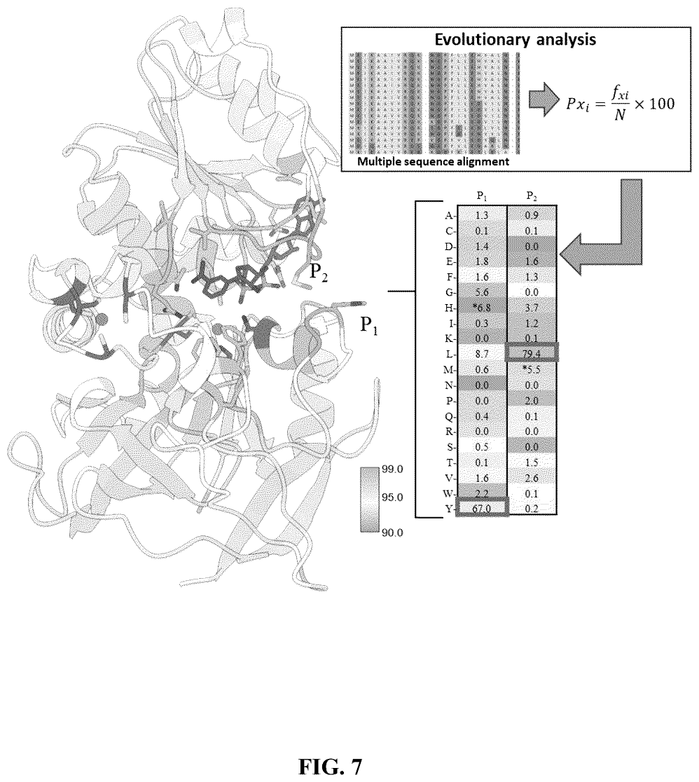

Continuous Learning: As more experimental data accumulates, it's fed back into the system to continually enhance the predictive accuracy of pLDDT scores FIG. 6 describes the sequential steps of P-POP and FIG. 7 depicts an example of the choice of hotspot and substitution using the P-POP protocol

### Engineered Monooxygenase

Engineered monooxygenase derived from the P-POP protocol as described in the previous steps corresponds to a polypeptide sequence that is at least 90% identical to the polypeptide given in SEQ ID 13-24, and that includes the feature of residue corresponding to X142 is Thr and additionally includes at least one or more of the following features: The residue corresponding to X19 is glycine, valine, serine, threonine, alanine, arginine, cysteine, lysine, or histidine; The residue corresponding to X27 is glycine, valine, serine, threonine, alanine, arginine, cysteine, lysine, or histidine; The residue corresponding to X28 is tryptophan, tyrosine, phenylalanine, serine, threonine, cysteine, alanine, aspartate, or glutamate; The residue corresponding to X29 is leucine, isoleucine, valine, serine, threonine, cysteine, alanine, aspartate, or glutamate; The residue corresponding to X31 is serine, threonine, cysteine, alanine, aspartate, or glutamate; The residue corresponding to X50 is serine, threonine, cysteine, alanine, aspartate, or glutamate; The residue corresponding to X55 is leucine, isoleucine, valine, alanine, phenylalanine, or proline; The residue corresponding to X77 is valine, alanine, aspartate or glutamate; The residue corresponding to X86 is aspartate, glutamate, arginine, or lysine; The residue corresponding to X89 is aspartate, glutamate, arginine, or lysine; The residue corresponding to X95 is glycine, lysine, serine, threonine, cysteine, alanine, aspartate, or glutamate; The residue corresponding to X98 is leucine, isoleucine, valine, lysine, arginine or alanine; The residue corresponding to X101 is serine, threonine, cysteine, alanine, aspartate, or glutamate; The residue corresponding to X109 is leucine, isoleucine, valine, alanine, histidine, methionine, threonine, serine, lysine, asparagine, or glutamine; The residue corresponding to X110 is leucine, isoleucine, valine, alanine, threonine, serine, lysine, arginine or proline; The residue corresponding to X123 is proline, aspartate, or glutamate; The residue corresponding to X125 is leucine, isoleucine, valine, alanine, threonine, serine, lysine, arginine, cysteine, or glycine; The residue corresponding to X128 is leucine, isoleucine, valine, alanine, histidine, methionine, threonine, serine, lysine, asparagine, or glutamine; The residue corresponding to X135 is glycine, serine, threonine, cysteine, alanine, aspartate, or glutamate; The residue corresponding to X140 is glycine, serine, threonine, cysteine, alanine, aspartate, or glutamate; The residue corresponding to X150 is tryptophan, tyrosine, phenylalanine, aspartate, or glutamate; The residue corresponding to X155 is leucine, isoleucine, valine, alanine, lysine or arginine; The residue corresponding to X177 is glycine, serine, threonine, cysteine, alanine, aspartate, or glutamate; The residue corresponding to X186 is glycine, valine, serine, threonine, alanine, arginine, cysteine, lysine, or histidine; The residue corresponding to X196 is proline, aspartate, or glutamate; The residue corresponding to X221 is glycine, valine, serine, threonine, alanine, arginine, cysteine, lysine, or histidine; The residue corresponding to X233 is leucine, isoleucine, valine, alanine, histidine, methionine, threonine, serine, or lysine; The residue corresponding to X235 is leucine, isoleucine, valine, lysine, arginine or alanine; The residue corresponding to X240 is leucine, isoleucine, valine, alanine, histidine, methionine, threonine, tyrosine, phenylalanine, serine, lysine, asparagine, or glutamine; The residue corresponding to X243 is tryptophan, tyrosine, phenylalanine, glycine, valine, serine, threonine, alanine, arginine, cysteine, lysine, or histidine; The residue corresponding to X244 is leucine, isoleucine, valine, alanine, histidine, asparagine, glutamine, phenylalanine, tyrosine, or tryptophan; The residue corresponding to X247 is arginine, glycine, glutamine, leucine, isoleucine, valine, serine, threonine, alanine, lysine, or asparagine; The residue corresponding to X250 is glycine, serine, threonine, alanine, aspartate, or glutamate; The residue corresponding to X252 is valine, leucine, isoleucine, alanine, aspartate, or glutamate; The residue corresponding to X255 is alanine, arginine, glutamine, leucine, isoleucine, lysine, proline, threonine, valine, or serine; The residue corresponding to X257 is glutamine, asparagine, alanine, glycine, serine, threonine, or lysine; The residue corresponding to X262 is histidine, aspartate, or glutamate; The residue corresponding to X264 is alanine, serine, threonine, valine, glycine, lysine or arginine; The residue corresponding to X267 is histidine, aspartate, or glutamate; The residue corresponding to X274 is proline, asparagine, aspartate, or glutamate; The residue corresponding to X276 is arginine, glycine, glutamine, leucine, isoleucine, valine, serine, threonine, alanine, lysine, or asparagine; The residue corresponding to X277 is cysteine, arginine, lysine, aspartate, asparagine, glutamate or glutamine; The residue corresponding to X279 is leucine, isoleucine, valine, alanine, glycine, phenylalanine, tyrosine, or tryptophan; The residue corresponding to X281 is alanine, valine, isoleucine, leucine, asparagine, glutamine, serine or threonine; The residue corresponding to X282 is histidine, aspartate, or glutamate; The residue corresponding to X293 is aspartate, cysteine, lysine, phenylalanine or tyrosine; The residue corresponding to X297 is arginine, lysine, phenylalanine, tyrosine or tryptophan; The residue corresponding to X308 is leucine, isoleucine, valine, alanine, arginine, lysine, aspartate, or glutamate; The residue corresponding to X337 is tyrosine, phenylalanine, tryptophan, lysine or arginine; The residue corresponding to X345 is leucine, isoleucine, valine, alanine, arginine, or lysine; The residue corresponding to X350 is asparagine, glutamine, serine, threonine, cysteine, or alanine; The residue corresponding to X355 is phenylalanine, tryptophan, tyrosine, serine, threonine or cysteine;

### Engineered Benzaldehyde Dehydrogenase

Engineered benzaldehyde dehydrogenases derived from the P-POP protocol as described in step 4 corresponds to a polypeptide sequence that is at least 90% identical to the polypeptide given in SEQ ID 26-33, and that includes the feature of residue corresponding to X105 is Arg or Lys and additionally includes at least one or more of the following features:

The residue corresponding to X9 is isoleucine, arginine, leucine, valine, alanine, lysine or histidine; The residue corresponding to X10 is tryptophan, cysteine, serine, threonine, alanine, glycine, phenylalanine, valine, tyrosine or methionine; The residue corresponding to X14 is valine, cysteine, isoleucine, leucine, alanine, serine, threonine, glycine, or methionine; The residue corresponding to X18 is asparagine, glycine, alanine, serine, threonine, glutamine, or aspartate; The residue corresponding to X26 is valine, glutamate, asparagine, glycine, alanine, serine, threonine, glutamine, glutamate, or aspartate; The residue corresponding to X28 is asparagine, glutamate, asparagine, glycine, alanine, serine, threonine, glutamine, glutamate, or aspartate; The residue corresponding to X37 is isoleucine, arginine, leucine, valine, alanine, lysine or histidine; The residue corresponding to X40 is isoleucine, lysine, leucine, valine, alanine, arginine or histidine; The residue corresponding to X42 is valine, cysteine, isoleucine, leucine, alanine, serine, threonine, glycine, or methionine; The residue corresponding to X43 is isoleucine, arginine, leucine, valine, alanine, lysine or histidine; The residue corresponding to X44 is alanine, cysteine, serine, threonine, glycine, proline or histidine; The residue corresponding to X64 is alanine, cysteine, serine, threonine, glycine, proline or histidine; The residue corresponding to X68 is tryptophan, cysteine, serine, threonine, alanine, glycine, phenylalanine, valine, tyrosine or methionine; The residue corresponding to X87 is tryptophan, cysteine, serine, threonine, alanine, glycine, phenylalanine, valine, tyrosine, aspartate, glutamate, asparagine or methionine; The residue corresponding to X122 is alanine, cysteine, serine, threonine, glycine, proline or histidine; The residue corresponding to X129 is valine, glutamate, asparagine, glycine, alanine, serine, threonine, glutamine, glutamate, leucine, isoleucine or aspartate; The residue corresponding to X140 is isoleucine, arginine, leucine, valine, alanine, lysine or histidine; The residue corresponding to X148 is isoleucine, arginine, leucine, valine, alanine, lysine or histidine; The residue corresponding to X155 is tryptophan, aspartate, glutamine, glycine, proline, serine, threonine, alanine, asparagine, glutamate, cysteine, phenylalanine, tyrosine or valine; The residue corresponding to X161 is leucine, asparagine, aspartate, isoleucine, methionine, glutamine, glycine, proline, serine, threonine, alanine, glutamate, cysteine, or valine; The residue corresponding to X173 is glycine, cysteine, serine, threonine, alanine, valine, or methionine; The residue corresponding to X177 is isoleucine, aspartate, leucine, valine, alanine, glutamate or asparagine; The residue corresponding to X178 is isoleucine, arginine, leucine, valine, alanine, lysine or histidine; The residue corresponding to X190 is glycine, cysteine, serine, threonine, alanine, valine, or methionine; The residue corresponding to X206 is isoleucine, aspartate, leucine, valine, alanine, glutamate or asparagine; The residue corresponding to X209 is leucine, cysteine, isoleucine, valine, alanine, serine, threonine, glycine or proline The residue corresponding to X218 is serine, threonine, alanine, lysine, glycine, valine, arginine, histidine or proline; The residue corresponding to X225 is isoleucine, aspartate, leucine, valine, alanine, glutamate or asparagine; The residue corresponding to X274 is serine, glutamate, aspartate, asparagine, threonine, glycine, valine, alanine or cysteine; The residue corresponding to X317 is isoleucine, arginine, leucine, valine, alanine, lysine or histidine; The residue corresponding to X323 is aspartate, glutamine, glutamate, asparagine, serine or threonine; The residue corresponding to X352 is glutamine, arginine, asparagine, lysine, histidine, serine or cysteine; The residue corresponding to X365 is aspartate, glutamine, glutamate, asparagine, serine or threonine; The residue corresponding to X380 is lysine, phenylalanine, arginine, tryptophan, tyrosine, or histidine; The residue corresponding to X381 is serine, glutamine, threonine, cysteine, asparagine or aspartate; The residue corresponding to X383 is isoleucine, cysteine, valine, methionine, histidine, leucine, alanine, serine or threonine; The residue corresponding to X385 is glycine, histidine, methionine, proline, valine, alanine, cysteine, serine, threonine, or lysine; The residue corresponding to X432 is serine, glutamine, threonine, cysteine, glycine, asparagine, glutamate or aspartate; The residue corresponding to X436 is isoleucine, arginine, leucine, valine, alanine, lysine or histidine; The residue corresponding to X443 is cysteine, leucine, phenylalanine, proline, serine, threonine, isoleucine, tyrosine, tryptophan, histidine, alanine or valine; The residue corresponding to X449 is phenylalanine, aspartate, tyrosine, tryptophan, glutamate, asparagine, or glutamine; The residue corresponding to X451 is glycine, arginine, lysine, alanine, histidine, serine or threonine; The residue corresponding to X461 is phenylalanine, isoleucine, lysine, leucine, arginine, tyrosine, tryptophan, or valine; The residue corresponding to X462 is glycine, asparagine, alanine, serine, threonine, glutamine, or aspartate; The residue corresponding to X465 is alanine, glutamine, serine, asparagine, threonine; glycine or aspartate; The residue corresponding to X472 is glutamine, glutamate, asparagine, aspartate, serine, threonine, or alanine; The residue corresponding to X475 is lysine, phenylalanine, arginine, tryptophan, tyrosine, or histidine; The residue corresponding to X476 is isoleucine, aspartate, leucine, valine, alanine, glutamate or asparagine; The residue corresponding to X483 is alanine, glutamate, tyrosine, phenylalanine, tryptophan, serine, threonine, valine or glycine; The residue corresponding to X484 is asparagine, arginine, aspartate, glutamine, glutamate, lysine, histidine, serine, threonine or tyrosine;

### Engineered Benzyl-Alcohol Dehydrogenase

The engineered benzyl alcohol dehydrogenase of claim 1 that is at least 90% identical to the polypeptide given in SEQ ID 35-40, derived from the polypeptide sequence mentioned in the SEQ ID 34, which is the gene product of the “B” gene as given by SEQ ID 4 and that includes the feature of the residue corresponding to X72 is Arg or Ser and additionally contains the following features:

The residue corresponding to X23 is, asparagine, arginine, lysine, glutamine, or aspartate; The residue corresponding to X27 is, glutamate, alanine, glycine, serine, threonine, aspartate, asparagine, glutamine, or valine; The residue corresponding to X36 is, alanine, arginine, serine, threonine, glycine, lysine, or valine; The residue corresponding to X38 is, alanine, arginine, serine, threonine, glycine, lysine, or valine; The residue corresponding to X45 is, valine, arginine, tryptophan, lysine, leucine, isoleucine, phenylalanine, or tyrosine; The residue corresponding to X46 is, cysteine, arginine, tyrosine, tryptophan, phenylalanine, serine, threonine, or lysine; The residue corresponding to X52 is, proline, glycine, isoleucine, threonine, serine, leucine, valine, or alanine; The residue corresponding to X73 is, alanine, histidine, serine, threonine, glycine, or valine; The residue corresponding to X75 is, lysine, glutamate, arginine, aspartate, asparagine, glutamine, or histidine; The residue corresponding to X99 is, glycine, aspartate, serine, threonine, alanine, valine, glutamate, or asparagine; The residue corresponding to X112 is, phenylalanine, tyrosine, tryptophan, or histidine; The residue corresponding to X118 is, threonine, arginine, serine, lysine, or alanine; The residue corresponding to X123 is, isoleucine, histidine, leucine, valine, phenylalanine, tryptophan, tyrosine, or alanine; The residue corresponding to X124 is, histidine, aspartate, glutamate, lysine, arginine, or asparagine; The residue corresponding to X126 is, histidine, alanine, cysteine, serine, threonine, glycine, or methionine; The residue corresponding to X127 is, glutamine, alanine, aspartate, asparagine, glycine, glutamate, serine, or threonine; The residue corresponding to X128 is, glycine, leucine, lysine, alanine, valine, isoleucine, serine, or threonine; The residue corresponding to X132 is, glycine, serine, threonine, alanine, cysteine, or valine; The residue corresponding to X133 is, glycine, serine, threonine, alanine, cysteine, or valine; The residue corresponding to X137 is, glycine, serine, threonine, alanine, cysteine, valine, asparagine, aspartate, glutamine, or glutamate; The residue corresponding to X138 is, glycine, serine, threonine, alanine, cysteine, valine, asparagine, aspartate, glutamine, or glutamate; The residue corresponding to X175 is, glycine, serine, threonine, alanine, cysteine, or valine; The residue corresponding to X179 is, leucine, glutamate, isoleucine, valine, aspartate, asparagine, glutamine, or alanine; The residue corresponding to X189 is, alanine, glutamate, valine, aspartate, asparagine, glutamine, serine, or threonine; The residue corresponding to X204 is, methionine, aspartate, asparagine, glutamine, glutamate, lysine, or alanine; The residue corresponding to X205 is, glycine, serine, threonine, alanine, cysteine, or valine; The residue corresponding to X206 is, alanine, lysine, arginine, serine, threonine, or valine; The residue corresponding to X207 is, glycine, serine, threonine, alanine, cysteine, or valine; The residue corresponding to X211 is, glycine, serine, threonine, alanine, cysteine, valine, asparagine, aspartate, glutamine, or glutamate; The residue corresponding to X213 is, glycine, serine, threonine, alanine, cysteine, valine, asparagine, aspartate, glutamine, or glutamate; The residue corresponding to X224 is, leucine, cysteine, serine, threonine, isoleucine, methionine, valine, or alanine; The residue corresponding to X227 is, glycine, serine, threonine, alanine, cysteine, or valine; The residue corresponding to X230 is, leucine, arginine, isoleucine, valine, or lysine; The residue corresponding to X231 is, glycine, serine, threonine, alanine, cysteine, or valine; The residue corresponding to X232 is, glycine, serine, threonine, alanine, cysteine, or valine; The residue corresponding to X235 is, leucine, cysteine, serine, threonine, isoleucine, methionine, valine, or alanine; The residue corresponding to X240 is, alanine, lysine, arginine, serine, threonine, or valine; The residue corresponding to X241 is, lysine, glutamate, arginine, aspartate, asparagine, glutamine, or histidine; The residue corresponding to X251 is, phenylalanine, arginine, tyrosine, lysine, tryptophan, or histidine; The residue corresponding to X252 is, alanine, glutamate, isoleucine, leucine, valine, aspartate, asparagine, or glutamine; The residue corresponding to X253 is, aspartate, phenylalanine, glutamate, tyrosine, asparagine, tryptophan, glutamine, or histidine; The residue corresponding to X256 is, proline, isoleucine, lysine, valine, leucine, alanine, arginine, or glycine; The residue corresponding to X275 is, glycine, serine, threonine, alanine, cysteine, or valine; The residue corresponding to X279 is, glycine, serine, threonine, alanine, cysteine, or valine; The residue corresponding to X286 is, alanine, asparagine, histidine, threonine, serine, aspartate, or valine; The residue corresponding to X301 is, leucine, histidine, tyrosine, isoleucine, phenylalanine, valine, or tryptophan; The residue corresponding to X310 is, leucine, histidine, tyrosine, isoleucine, phenylalanine, valine, or tryptophan; The residue corresponding to X311 is, aspartate, phenylalanine, glutamate, tyrosine, asparagine, tryptophan, glutamine, or histidine; The residue corresponding to X313 is, glutamine, glutamate, asparagine, aspartate, serine, or threonine; The residue corresponding to X315 is, isoleucine, arginine, leucine, lysine, valine, or histidine; The residue corresponding to X326 is, leucine, arginine, cysteine, isoleucine, lysine, serine, valine, alanine, or threonine; The residue corresponding to X332 is, phenylalanine, cysteine, tryptophan, serine, threonine, tyrosine, or alanine; The residue corresponding to X350 is, glycine, serine, threonine, alanine, cysteine, valine, asparagine, aspartate, glutamine, or glutamate;

### Optimized Synthesis of the Mutants:

An avant-garde approach was embraced to generate coding sequences tailored for optimum expression within the Escherichia coli environment. These synthetic gene constructs were ingeniously fabricated using a one-pot reaction system, ensuring efficiency and precision. The process necessitates the set of four genes, each responsible for expressing specific enzymes vital for converting 3-methylpyridine to nicotinic acid. The gene responsible for benzyl alcohol dehydrogenase production, represented by SEQ ID No. 2, can be sidestepped, as needed, to counteract potential back-conversion due to product inhibition.

### Design and Strategy:

The operational blueprint was architected with the primary objective of co-expressing these genes in one cellular unit. Three cornerstone genes-“M”, “A”, and “C”-were methodically extracted from strains such as Pseudomonas putida pWWO and Pseudomonas putida F1. To strengthen expression efficiency, each synthetic construct was embedded with ribosomal binding sites (RBS) and spacers, each meticulously engineered with an optimal number of bases.

### Vector Systems and Constructs:

Central to this methodology is the versatile pET28a(+) vector, identified as the paragon for gene expression within the E. coli matrix. However, a suite of alternative vectors like pRSFDuet-1, pCDFDuet-1, and pETDuet-1 were also devised to create diverse gene constructs. The pET28a(+) plasmid vector emerged as a primary choice due to its demonstrated efficiency and reliability. Explorative attempts were made to design fusion proteins, ensuring each protein retains its distinct identity yet is co-expressed within the same cellular confines. To achieve this, strategic placement of stop codons after each gene sequence was done, followed by a spacer and the ATG start codon of the subsequent protein.

### Restriction Enzymes and Cloning Facets:

Despite the synthetic origins of these genes, the importance of restriction enzyme sites, particularly for future cloning endeavours, was pivotal. A discovery was made of the XhoI site within the “A” gene of the pWWO variant. Thus, the unique NcoI site, encompassing the starting ATG, was harmonized with the NotI site, ensuring neither appeared within the three primary genes, for optimal cloning pursuits.

### Expression Concerns and Strategy:

To prevent potential hindrances in expression levels caused by his-tags, genes were cloned into the NcoI and XhoI sites with a preceding stop codon to XhoI, negating 3′ his-tags. Diverse combinations were formulated with the pET28a(+) vector, encompassing multiple gene orderings and origins. While fusion protein combinations were explored, concerns regarding protein folding led to the inclusion of a stop codon post each gene, followed by the start codon for the subsequent protein.

### Synthesis and Cloning:

Given that genes were synthesized, restriction enzyme site considerations were omitted for the primary construct. Nonetheless, future cloning endeavours necessitate the examination of RE within genes for streamlined cloning procedures. Unique site considerations, like the presence of XhoI within the “A” gene of pWWO, were addressed by opting for NcoI (incorporating the starting ATG) combined with NotI (a distinct site absent in the three primary genes).

### Detailed Construct Outlines:

Various constructs were meticulously designed with specific plasmid vectors, each encompassing distinct genes and sequences from P. putida genomes. Each construct emphasizes efficient expression through the inclusion of RBS sequences preceding the respective open reading frame (ORF) of individual genes.

### Construct Specifications

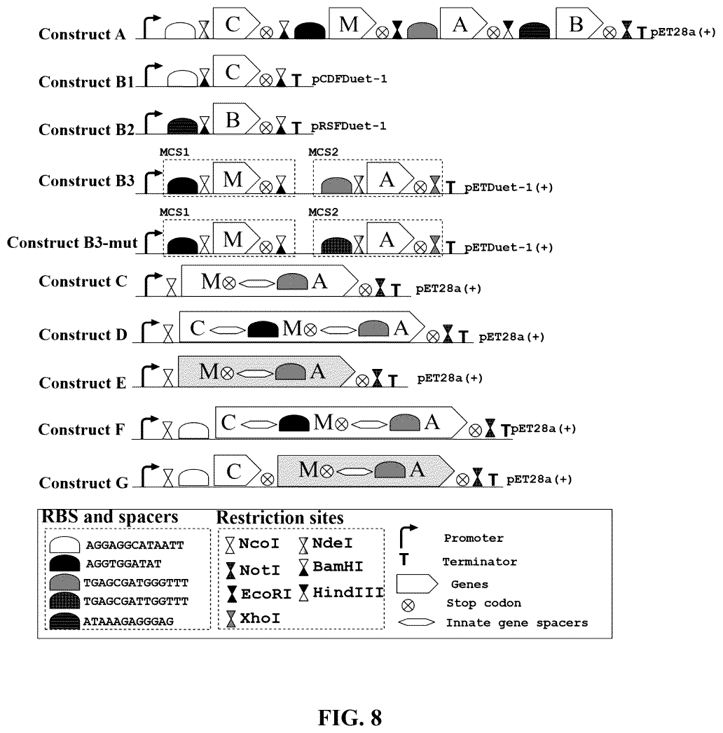

Multiple constructs were generated, each harbouring distinct genes, RBS sites, and specific restriction enzyme sites, all optimized for maximum efficiency (FIG. 8).

“Construct A” is designed using a pET28a(+) plasmid vector, harbouring “C” gene as given by SEQ ID No. 3, “M” gene as given by SEQ ID No. 1, “A” gene as given by SEQ ID No. 2, and “B” gene as given by SEQ ID No. 4, in that respective order, expressed with short ribosomal binding and spacer (RBS) sequences upstream to the open reading frame (ORF) of the individual genes to improve the expression of the genes in between the NcoI and NotI restriction sites; wherein the RBS sequence expressed upstream to “C” gene is given in SEQ ID No. 7; the RBS sequence expressed upstream to “M” gene is given in SEQ ID No. 8; the RBS sequence expressed upstream to “A” gene is given in SEQ ID No. 9; the RBS sequence expressed upstream to “B” gene is given in SEQ ID No. 10.

“Construct B1” is designed using a pCDFDuet-1 plasmid vector harbouring “C” as given by SEQ ID No. 3, expressed with short ribosomal binding and spacer (RBS) sequence upstream to the open reading frame (ORF) of the individual gene to improve the expression of the gene expressed in between the NcoI and BamHI restriction sites; wherein the RBS sequence expressed upstream to “C” gene is given in SEQ ID No. 7.

“Construct B2” is designed using a pRSFDuet-1 plasmid vector, harbouring “B” as given by SEQ ID No. 2, expressed with short ribosomal binding and spacer (RBS) sequence upstream to the open reading frame (ORF) of the individual gene to improve the expression of the gene in between the NcoI and BamHI restriction sites; wherein the RBS sequence expressed upstream to “B” gene is given in SEQ ID No. 10.

“Construct B3” is designed using a pETDuet-1 plasmid vector, harbouring “M” as given by SEQ ID No. 1, and “A” as given by SEQ ID No. 2, in that respective order are expressed with short ribosomal binding and spacer (RBS) sequences upstream to the open reading frame (ORF) of the individual genes to improve the expression of the genes in between the NcoI and XhoI restriction sites; wherein the RBS sequence expressed upstream to “M” gene is given in SEQ ID No. 8; the RBS sequence expressed upstream to “A” gene is given in SEQ ID No. 9.

“Construct B3-mut” is designed using a pETDuet-1 plasmid vector, harbouring “M” as given by SEQ ID No. 1, and “A” as given by SEQ ID No. 2, in that respective order are expressed with short ribosomal binding and spacer (RBS) sequences upstream to the open reading frame (ORF) of the individual genes to improve the expression of the genes in between the NcoI and XhoI restriction sites; wherein the RBS sequence expressed upstream to “M” gene is given in SEQ ID No. 8; the RBS sequence expressed upstream to “A” gene is given in SEQ ID No. 11. This construct differs from “Construct-B3” by a point mutation in the spacer between the RBS sequence expressed upstream to “A” gene.

“Construct C” is designed using a pET28a(+) plasmid vector harbouring the genes wherein the gene was taken from downstream of the start codon of the monooxygenase gene (“M”) to the termination codon of the electron transfer component gene of the monooxygenase gene (“A”) of genome of P. putida pWWO organism. This gene component contains endogenous gene fragments such as the RBS and the spacer for the expression of the “A” genes that are innate to the genome and are present in the spacer region between mono oxygenase and the electron transfer component gene. The gene component was expressed in between the NcoI and NotI restriction sites.

“Construct D” is designed using a pET28a(+) plasmid vector harbouring the genes wherein the gene was taken from downstream of the start codon of the benzaldehyde dehydrogenase (“C”) to the termination codon of the electron transfer component gene of the monooxygenase gene (“A”) of genome of P. putida pWWO organism. This gene component contains endogenous gene fragments such as the RBS and the spacer for the expression of the “M” and “A” genes that are innate to the genome and are present in the spacer region between the ORF of benzaldehyde dehydrogenase & monooxygenase and mono oxygenase and the electron transfer component gene, respectively. The gene component was expressed in between the NcoI and NotI restriction sites.

“Construct E” is designed using a pET28a(+) plasmid vector harbouring the genes wherein the gene was taken from downstream of the start codon of the monooxygenase gene (“M” to the termination codon of the electron transfer component gene of the monooxygenase gene (“A”) of genome of P. putida F1 organism. This gene component contains endogenous gene fragments such as the RBS and the spacer for the expression of the “A” genes that are innate to the genome and are present in the spacer region between mono oxygenase and the electron transfer component gene. The gene component was expressed in between the NcoI and NotI restriction sites.

“Construct F” is designed using a pET28a(+) plasmid vector harbouring the genes wherein the gene was taken from downstream of the start codon of the benzaldehyde dehydrogenase (“C”) to the termination codon of the electron transfer component gene of the monooxygenase gene (“A”) of the genome of P. putida pWWO. This gene component contains endogenous gene fragments such as the RBS and the spacer for the expression of the “M” and “A” genes that are innate to the genome and are present in the spacer region between the ORF of benzaldehyde dehydrogenase & monooxygenase and mono oxygenase and the electron transfer component gene, respectively. The gene component was expressed in between the NcoI and NotI restriction sites. Additionally, the construct also includes an optimized RBS and spacer sequence positioned upstream of the start codon of the “C” gene in the genome component.

“Construct G” is designed using a pET28a(+) plasmid vector harbouring the genes wherein the gene was taken from downstream of the start codon of the monooxygenase gene (“M” to the termination codon of the electron transfer component gene of the monooxygenase gene (“C”) of genome of P. putida F1 organism. This gene component contains endogenous gene fragments such as the RBS and the spacer for the expression of the “A” genes that are innate to the genome and are present in the spacer region between mono oxygenase and the electron transfer component gene. Additionally, the construct also houses “C” gene, the RBS and spacer for expressing the “C” gene in the same construct. The gene component was expressed in between the NcoI and NotI restriction sites.

Immobilization of the whole cell catalyst: One significant aspect of this invention is the immobilization of the whole cell catalyst. Industrial-scale applications employing recombinant E. coli as a whole-cell catalyst are often met with challenges, including the fragility of the cells, mechanical damages, and fluctuations in pH and thermal stability. These obstacles can impede the reusability of the whole-cell catalyst, consequently escalating the overall costs of industrial production by necessitating the continual replacement of the whole-cell catalyst's fresh biomass. To mitigate these challenges, techniques such as encapsulation or entrapment of biocatalysts like enzymes and whole cell catalysts are integrated. These biocatalysts can be embedded within a biocompatible, water insoluble, crosslinked polymer matrix derived from either natural or synthetic linear polysaccharides. Upon interaction with divalent or trivalent metal ions, these matrices can morph into hydrogels, such as those formed from calcium alginate or calcium K-carrageenan. A straightforward methodology employed for immobilization involves dripping a combination of the cell suspension and sodium alginate solution into a calcium chloride solution. The ensuing hydrogel formation not only ensures the viability of the encapsulated cells but also allows accessibility to the requisite reactants. This method provides myriad advantages, notably the reusability of the catalyst, heightened stability, and shielding against mechanical damage. Furthermore, immobilizing whole cell catalysts can prolong their storage life, preserving their viability for up to 60 days in refrigerated conditions. For the actual immobilization process, the invention employs a specific technique. Recombinant E. coli cells are immobilized using the CaCl₂-Sodium alginate method, wherein 120 mg of engineered cells (wet weight) are combined with a 4 mL sodium alginate solution (2.5%, w/v). This mixture is incrementally added to a 2% (w/v) CaCl₂ solution. Post-hardening, the resultant beads are rinsed with a Tris-HCl buffer to yield calcium alginate-immobilized cells.

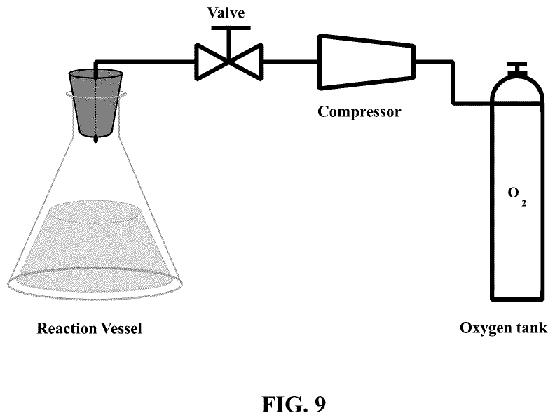

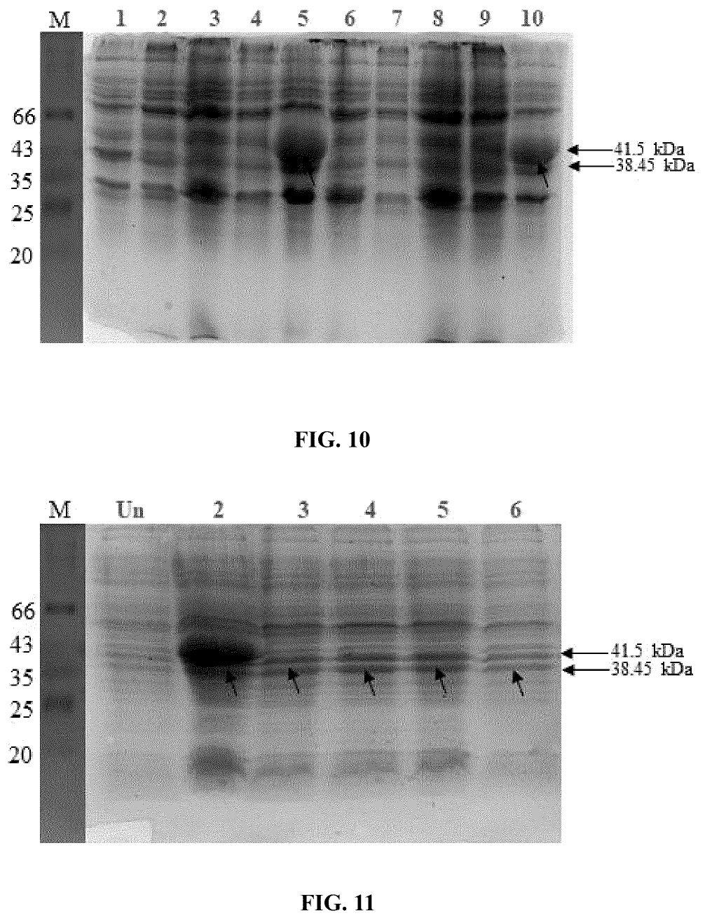

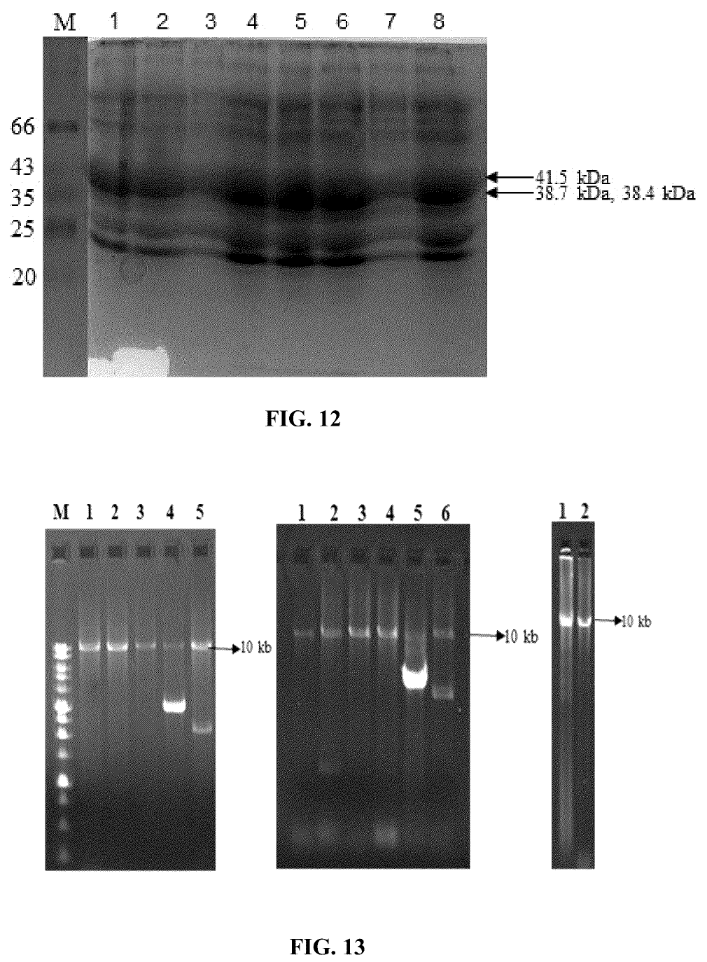

External oxygen supply: A pivotal component of this invention is the integration of oxygen. Considering that monooxygenases utilize oxygen molecules as electron donors, the reactions they facilitate are primarily driven by oxygen. Direct oxygen incorporation can markedly amplify the biocatalytic efficiency of oxygen-dependent reactions compared to using augmented enzyme biocatalyst quantities. Due to the inherently low oxygen concentration in aqueous systems, larger scales can underscore oxygen transfer limitations. To address this, the invention introduces pure oxygen directly to a reaction vessel containing the reaction mixture. The vessel's design incorporates a one-way valve that permits oxygen from a connected chamber to access the reaction mixture (FIG. 9).

Cell permeability: The invention emphasizes enhancing cell permeability, which is paramount for whole-cell catalysis. To ensure efficient enzymatic reactions, substrates must easily permeate into the cytosol, where most engineered enzyme catalysts are expressed. To improve this permeability, the invention uses Tween 80 and TritonX, a non-ionic detergent capable of creating substantial pores in the plasma membrane without compromising its structural integrity. Through the incorporation of 0.2% Tween-20 and a 30-minute incubation period, the invention achieves optimal results in cell permeability.

### Example 1

### Expression of Enzymes of the MO System

The plasmid containing strains of E. coli BL21 (DE3) cells housing any of the constructs A-G with the wild genes or the engineered genes generated by SDM was incubated for 12 h at 37° C. on a rotary shaker (220 rpm) in Luria Bertani (LB) medium with ampicillin, kanamycin, and streptomycin. Thereafter, 1% [vol/vol] of the seed culture was added to Terrific Broth (TB) medium with the same antibiotics and cultivated at 37° C. and 220 rpm. When the optical density at 600 nm (OD₆₀₀) reached 1.2, isopropyl-β-D-thiogalactopyranoside (IPTG) was immediately added to the broth to a final concentration of 0.05 mM. After 10 h of incubation at 28° C., cells were harvested by centrifugation at 10,000×g for 10 min at 4° C. The pellets were washed twice with sterilized water, and then were suspended in the Na₂HPO₄—NaH₂PO₄ buffer and kept at 4° C. until further use.

### Example 2

Whole-cell catalysis Assay was conducted for all the construct and variants using the following protocol. Biomass was quantified by measuring OD₆₀₀ and converted to dry cell weight (DCW) using the following equation: DCW (g/L)=0.4442×OD₆₀₀−0.021

For assays of whole-cell biocatalytic activity, a mixture of whole-cell biocatalyst (E. coli BL21 (DE3) cells housing any of the constructs with the wild genes or the engineered genes generated by SDM and 4 g/L substrate was incubated in Erlenmeyer flasks (50 ml) at 220 rpm and 30° C. for 24 h. The reaction mixture (10 ml) was made with 200 mM Na₂HPO₄—NaH₂PO₄ buffer (pH 7.0). Reactions were stopped by centrifugation at 10,000×g for 10 min and the supernatant was then analyzed by HPLC.

TABLE 1
Comparative analysis of product formation
across various constructs.

| | | Dry cell | Percentage | Fold |
|---|---|---|---|---|
| S. No. | Construct/Variant | Weight (g) | conversion | increase |
| 1 | Construct A | 1 | 0.415 | 0 |
| 2 | Construct B | 1 | 0.421 | 1.0 |
| 3 | Construct B | 0.5 | 0.473 | 1.1 |
| 4 | Construct D | 1 | 0.483 | 1.2 |
| 5 | Construct B | 1 | 0.512 | 1.2 |
| 6 | M1_R1_1 | 0.4 | 59.689 | 143.8 |
| 7 | M1_R1_12 | 0.4 | 55.456 | 133.6 |
| 8 | M1_R1_41 | 0.4 | 44.923 | 108.2 |
| 9 | M1_R1_4 | 0.4 | 40.312 | 97.1 |
| 10 | M1_R1_3 | 0.4 | 40.132 | 96.7 |
| 11 | M1_R1_9 | 0.4 | 39.156 | 94.4 |
| 12 | M1_R1_10 | 0.4 | 39.132 | 94.3 |
| 13 | M1_R1_15 | 0.4 | 38.198 | 92.0 |
| 14 | M1_R1_13 | 0.4 | 20.154 | 48.6 |
| 15 | M3_R1_5 | 0.4 | 60.269 | 145.2 |
| 16 | M3_R1_2 | 0.4 | 40.378 | 97.3 |
| 17 | M3_R1_14 | 0.4 | 34.772 | 83.8 |
| 18 | M3_R1_8 | 0.4 | 32.252 | 77.7 |
| 19 | M1_R1_1 + M3_R1_5 | 0.4 | 74.689 | 180.0 |
| 20 | M1_R1_12 + M3_R1_2 | 0.4 | 69.896 | 168.4 |

MX_R1_N are constructs with engineered genes, where X is the identifier for the engineered gene (1=Monooxygenase and 3-Benzaldehyde dehydrogenase), N is the number of a particular mutant construct. The table showcases product formation under different dry cell weight concentrations and incubation conditions using multiple constructs, specifically Construct A, Construct B, and Construct D. The enhanced product yield observed with the engineered Construct-A variants is particularly notable. Each reaction was carried out using 0.4 g dry cell weight, equivalent to a concentration of 40 g/L. The engineering of the monooxygenase and benzaldehyde dehydrogenase was informed by insights obtained from an in silico engineering process. The “Fold increase” column demonstrates the relative enhancement in product formation when compared to the wild-type construct. The engineered enzyme variants displayed a 180-fold boost in enzyme activity with a mere dry cell weight of 0.4 grams. When the dry cell weight was increased to 4.2 grams, an impressive conversion rate of 99.5% for the substrate was achieved (Data not shown).

### Example 3

### Modification and Evaluation of the Monooxygenase Via in Silico-Guided Engineering

The monooxygenase underwent modification based on insights from an in-silico engineering process. Site-directed mutagenesis of the Construct A was executed utilizing pfu Taq DNA polymerase. The primer sequences employed for respective site-directed mutagenesis efforts can be referenced in the accompanying table. Polymerase Chain Reaction (PCR) parameters were as follows:
Initial denaturation: 98° C. for 1 minute.
Denaturation, annealing, and extension cycles (25 iterations): 98° C. for 10 seconds, 65° C. for 30 seconds, and 72° C. for 10 minutes respectively.
Final extension: 72° C. for 20 minutes.

Post-PCR, the resultant amplicons were subjected to 0.8% agarose gel electrophoresis for evaluation. Verified variants underwent Dpn1 digestion and subsequent transformation into the DH5α strains. Extracted plasmid DNA was then introduced into E. coli BL21 (DE3) to assess the protein expression profile, which was visualized on a 10% SDS-PAGE.

Notably, the modified enzyme variants demonstrated an enhanced enzymatic activity, showcasing a 23-fold increase at a dry cell weight of 0.4 grams. When assessed at a higher dry cell weight of 4.2 grams, the substrate conversion efficiency peaked at 99.5%.

### Example 4

### Influence of Oxygen on Product Formation:

For whole-cell biocatalytic assay, a mixture of whole-cell biocatalyst and 4 g/L substrate was incubated in Erlenmeyer flasks (50 ml) at 220 rpm and 30° C. for 24 h in the presence of oxygen. Supply the oxygen through the balloon and conduct the reaction under continuous stirring with 10 ml of 200 mM Na₂HPO₄—NaH₂PO₄ buffer (pH 7.0). Reactions were stopped by centrifugation at 10,000×g for 10 min and the supernatant was then analyzed by HPLC. The process showed ˜4% increase in the product formation compared to the wildtype.

### Example 5

Improvising cell permeability: For whole-cell biocatalytic assay, a mixture of whole-cell biocatalyst and 4 g/L substrate with 0.2% Triton X 100. Incubate the Erlenmeyer flasks (50 ml) at 220 rpm and 30° C. for 36 h and conduct the reaction under continuous stirring with 10 ml of 200 mM Na₂HPO₄—NaH₂PO₄ buffer (pH 7.0). Reactions were stopped by centrifugation at 10,000×g for 10 min and the supernatant was then analyzed by HPLC. The process showed 1.8% increase in the product formation compared to the wildtype.

### Advantages of the Invention

The invention offers a promising alternative to produce nicotinic acid, potentially bringing economic, environmental, and industrial benefits.

The method employs microbial biotransformation, a green chemistry approach, reducing reliance on chemical synthesis and minimizing environmental impacts. Utilizing byproducts like 3-picoline further contributes to ascribing value to waste and resource efficiency.

The strategic introduction of gene mutations and the assembly of diverse genes are aimed at producing mutated enzymes with improved catalytic properties. These enhancements are intended to optimize conversion rates and yields of nicotinic acid from 3-picoline.

Co-expression of all necessary enzymes in one host organism (E. coli) eliminates the need for multiple isolated reactions or microbial strains, simplifying production logistics. This approach also enables the direct and sequential transformation of 3-picoline to nicotinic acid, potentially improving the overall yield and reducing production times.

The invention's use of 3-picoline, an economically abundant and low-cost substrate, ensures a cost-effective production method for nicotinic acid. The potential scalability of this method could lead to a reduction in production costs, benefiting both manufacturers and consumers.

The advanced engineering of the proteins involved seeks to optimize enzyme performance and achieve high conversion rates, surpassing natural enzymatic capacities. The strategic optimization of individual enzymatic steps ensures high specificity and efficiency in the overall process.

Beyond synthesizing nicotinic acid, the evolved MO system can find applications in producing various other valuable compounds and in environmental remediation. The technology can be potentially modified or expanded to synthesize other related compounds or molecules of interest.

Nicotinic acid has well-documented therapeutic and nutritional benefits, including maintaining cholesterol balance and averting cardiovascular ailments.

Enhanced production of nicotinic acid can contribute to meeting the growing demands in pharmaceutical and nutraceutical industries, thereby promoting public health.

E. coli, the host organism, is known for its versatile metabolic capabilities and well-characterized genetic landscape, offering a robust and versatile platform for gene expression and metabolic engineering. The incorporation of genes from different microbial species allows for a meticulous characterization and subsequent validation of the efficacy of each construct in driving the conversion of 3-picoline to nicotinic acid.

The invention represents a novel intersection of synthetic biology, metabolic engineering, and enzyme optimization, pushing the boundaries of what is possible in biotechnological synthesis. It serves as a model for how synthetic biology and enzyme engineering can transform the synthesis of other compounds, driving innovations in various fields.

With the global nicotinic acid market having reached $614M in 2019, innovations in its production methods can have significant economic impacts. The invention could potentially capture a significant share of the market, given its advantages in sustainability, efficiency, and cost-effectiveness.

The biocatalytic approach offers a clean and potentially less energy intensive alternative to chemical synthesis methods. The use of microbial biotransformation contributes to environmental preservation by minimizing waste and reducing the emission of pollutants.

What is claimed is:

1. A method for the synthesis of nicotinic acid from 3-picoline, the method consisting of steps
a. Extracting genes “C” “M”, “A” and “B”, distinctly from any of the genomes of selected organisms such as Pseudomonas putida, Arthrobacter woluwensis, Acidovorax sp., Acinetobacter calcoaceticus, Burkholderia sp., Croceicoccus sp., Cupriavidus sp., Delftia sp., Devosia sp., Geodermatophilus sp., Jatrophihabitans sp., Kribella sp., Lacisediminimonas sp., Microbacterium sp., Mycolicibacterium sp., Nocardioides sp., Novosphingobium sp., Parapusillimonas sp., Planosporangium sp., Prauserella sp., Ramlibacter sp., Rhodococcus sp wherein, the genes “C”, “M”, “A” and “B” encode the proteins benzaldehyde dehydrogenase, monooxygenase, electron transfer component of the monooxygenase and benzyl alcohol dehydrogenase, respectively and the genes “C”, “M”, “A” and “B” include the associated genetic components such as RBS and spacers from the respective genome
b. Designing synthetic gene constructs with the extracted genes in the wild or engineered form, cloning the synthetic gene construct into an expression vector at specific restriction enzyme sites such as NcoI, NdeI, BamHI, EcoRI, HindIII, XhoI, and NotI; expressing these cloned genes within a transforming recombinant host cells, wherein the engineering of the genes are done involving site-directed mutagenesis, rational design, directed evolution, or a combination thereof.
c. Culturing the transformed host cells under conditions suitable for expression of said genes to convert 3-picoline to nicotinic acid via enzymatic action of expressed proteins from said genes.

2. The method of claim 1, wherein the engineered “C”, “M”, “A” and “B” gene product proteins exhibit enhanced performance characteristics like increased yield, improved stability and or enhanced catalytic efficiency as compared to the wild-type “C”, “M”, “A” and “B” gene products.

3. The method of claim 1, wherein the “C”, “M”, “A” and “B” genes corresponding to SEQ ID 1, 2, 3, 4, respectively sourced from the genome of Pseudomonas putida pWWO or “M”, “A” genes corresponding to SEQ ID 5, 6 are sourced from Pseudomonas putida F1 and wherein said expression vector is selected from the group consisting of pET28a(+), pRSFDuet-1, pCDFDuet-1, and pETDuet-1 and transforming recombinant host cells are selected from microorganisms such as Escherichia coli, Saccharomyces cerevisiae, Pichia pastoris, Schizosaccharomyces pombe, Aspergillus niger, Trichoderma reesei, and Penicillium chrysogenum.

4. The method of claim 3 wherein,
a. said plasmid vector is pET28a(+), and the genes in the synthetic construct are arranged in the order of “C” followed by “M” followed by “A” and optionally by “B” and wherein the RBS and spacer corresponding to “C” is given by SEQ ID 7, “M” is given by SEQ ID 8, “A” is given by SEQ ID 9 and “B” is given by SEQ ID 10, 11 and the recombinant DNA construct obtained thereof.
b. said plasmid vector is pCDFDuet-1, housing “C” gene in the synthetic construct and wherein the RBS and spacer corresponding to “C” is given by SEQ ID 7 and the recombinant DNA construct obtained thereof.
c. said plasmid vector is pRSFDuet-1, housing “B” gene in the synthetic construct and wherein the RBS and spacer corresponding to “B” is given by SEQ ID 10, 11 and the recombinant DNA construct obtained thereof.
d. said plasmid vector is pETDuet-1, and the genes in the synthetic construct are arranged in the order of “M” followed by “A” and wherein the RBS and spacer corresponding to “M” is given by SEQ ID 8, and “A” is given by SEQ ID 9 and the recombinant DNA construct obtained thereof.

5. A transforming recombinant host cell of claim 3 wherein the single transforming recombinant host cell expresses two or more gene constructs simultaneously wherein one vector houses the “B” gene, a second vector houses “C” gene, and a third vector houses “M” and “A” genes or wherein one vector houses “C” gene and a second vector houses “M” and “A” genes and wherein the “B” gene is optionally omitted to prevent back-conversion due to product inhibition.

6. A transforming recombinant host cell of claim 1 expressing two or more gene constructs simultaneously; wherein a vector housing “C” gene or the “B” gene and a vector housing:
a. The component of the genome was taken from downstream of the start codon of the monooxygenase gene in the genome of pWWO to the termination codon of the electron transfer component gene of the monooxygenase gene in the same genome of pWWO or F1 and which additionally includes the endogenous gene fragments such as the RBS and the spacer for the expression of the “A” gene that are innate to the genome, present in the spacer region between the ORF of monooxygenase and the electron transfer component gene
b. The component of the genome was taken from downstream of the start codon of the benzaldehyde dehydrogenase gene in the genome of pWWO of F1 to the termination codon of the electron transfer component gene of the monooxygenase gene in the same genome of pWWO or F1 and which additionally includes the endogenous gene fragments such as the RBS and the spacer for the expression of the “M” & “A” genes that are innate to the genome present in the two spacer regions between the ORF of benzaldehyde dehydrogenase gene, the monooxygenase gene and electron transfer component gene, respectively.

7. Engineered monooxygenase of claim 1 that is at least 90% identical to the polypeptide given in SEQ ID 13, derived from the polypeptide sequence mentioned in the Sequence ID 12, which is the gene product of the “M” gene as given by Sequence ID 1 and that includes the feature of residue corresponding to X142 is T.

8. The monooxygenase polypeptide of claim 7 comprising a polypeptide that is 90% identical to any of the amino acid sequences given in SEQ ID 14-24 and wherein the amino acid sequences additionally include at least one or more of the following features as detailed in the previously provided list.
The residue corresponding to X244 is an aspartate, a glutamine, a histidine, a leucine, or a phenylalanine residue.
The residue corresponding to X247 is an arginine, a leucine, a lysine, or a valine residue.
The residue corresponding to X86 is an arginine or a lysine residue.
The residue corresponding to X89 is an arginine or a lysine residue.
The residue corresponding to X276 is an alanine, a lysine, a glutamine, or a valine residue.
The residue corresponding to X279 is a glycine or a tyrosine residue.
The residue corresponding to X109 is an asparagine, a histidine, a methionine, a threonine, or a valine residue.
The residue corresponding to X123 is an aspartate or a glutamate residue.
The residue corresponding to X243 is an alanine, an arginine, or a serine residue.
The residue corresponding to X110 is an arginine, a leucine, a serine, a threonine, or a valine residue.
The residue corresponding to X240 is an alanine, an asparagine, a phenylalanine, or a tyrosine residue.
The residue corresponding to X19 is glycine, valine, serine, threonine, alanine, arginine, cysteine, lysine, or histidine;
The residue corresponding to X27 is glycine, valine, serine, threonine, alanine, arginine, cysteine, lysine, or histidine;
The residue corresponding to X28 is tryptophan, tyrosine, phenylalanine, serine, threonine, cysteine, alanine, aspartate, or glutamate;
The residue corresponding to X29 is leucine, isoleucine, valine, serine, threonine, cysteine, alanine, aspartate, or glutamate;
The residue corresponding to X31 is serine, threonine, cysteine, alanine, aspartate, or glutamate;
The residue corresponding to X50 is serine, threonine, cysteine, alanine, aspartate, or glutamate;
The residue corresponding to X55 is leucine, isoleucine, valine, alanine, phenylalanine, or proline;
The residue corresponding to X77 is valine, alanine, aspartate or glutamate;
The residue corresponding to X86 is aspartate, glutamate, arginine, or lysine;
The residue corresponding to X89 is aspartate, glutamate, arginine, or lysine;
The residue corresponding to X95 is glycine, lysine, serine, threonine, cysteine, alanine, aspartate, or glutamate;
The residue corresponding to X98 is leucine, isoleucine, valine, lysine, arginine or alanine;
The residue corresponding to X101 is serine, threonine, cysteine, alanine, aspartate, or glutamate;
The residue corresponding to X109 is leucine, isoleucine, valine, alanine, histidine, methionine, threonine, serine, lysine, asparagine, or glutamine;
The residue corresponding to X110 is leucine, isoleucine, valine, alanine, threonine, serine, lysine, arginine or proline;
The residue corresponding to X123 is proline, aspartate, or glutamate;
The residue corresponding to X125 is leucine, isoleucine, valine, alanine, threonine, serine, lysine, arginine, cysteine, or glycine;
The residue corresponding to X128 is leucine, isoleucine, valine, alanine, histidine, methionine, threonine, serine, lysine, asparagine, or glutamine;
The residue corresponding to X135 is glycine, serine, threonine, cysteine, alanine, aspartate, or glutamate;
The residue corresponding to X140 is glycine, serine, threonine, cysteine, alanine, aspartate, or glutamate;
The residue corresponding to X150 is tryptophan, tyrosine, phenylalanine, aspartate, or glutamate;
The residue corresponding to X155 is leucine, isoleucine, valine, alanine, lysine or arginine;
The residue corresponding to X177 is glycine, serine, threonine, cysteine, alanine, aspartate, or glutamate;
The residue corresponding to X186 is glycine, valine, serine, threonine, alanine, arginine, cysteine, lysine, or histidine;
The residue corresponding to X196 is proline, aspartate, or glutamate;
The residue corresponding to X221 is glycine, valine, serine, threonine, alanine, arginine, cysteine, lysine, or histidine;
The residue corresponding to X233 is leucine, isoleucine, valine, alanine, histidine, methionine, threonine, serine, or lysine;
The residue corresponding to X235 is leucine, isoleucine, valine, lysine, arginine or alanine;
The residue corresponding to X240 is leucine, isoleucine, valine, alanine, histidine, methionine, threonine, tyrosine, phenylalanine, serine, lysine, asparagine, or glutamine;
The residue corresponding to X243 is tryptophan, tyrosine, phenylalanine, glycine, valine, serine, threonine, alanine, arginine, cysteine, lysine, or histidine;
The residue corresponding to X244 is leucine, isoleucine, valine, alanine, histidine, asparagine, glutamine, phenylalanine, tyrosine, or tryptophan;
The residue corresponding to X247 is arginine, glycine, glutamine, leucine, isoleucine, valine, serine, threonine, alanine, lysine, or asparagine;
The residue corresponding to X250 is glycine, serine, threonine, alanine, aspartate, or glutamate;
The residue corresponding to X252 is valine, leucine, isoleucine, alanine, aspartate, or glutamate;
The residue corresponding to X255 is alanine, arginine, glutamine, leucine, isoleucine, lysine, proline, threonine, valine, or serine;
The residue corresponding to X257 is glutamine, asparagine, alanine, glycine, serine, threonine, or lysine;
The residue corresponding to X262 is histidine, aspartate, or glutamate;
The residue corresponding to X264 is alanine, serine, threonine, valine, glycine, lysine or arginine;
The residue corresponding to X267 is histidine, aspartate, or glutamate;
The residue corresponding to X274 is proline, asparagine, aspartate, or glutamate;
The residue corresponding to X276 is arginine, glycine, glutamine, leucine, isoleucine, valine, serine, threonine, alanine, lysine, or asparagine;
The residue corresponding to X277 is cysteine, arginine, lysine, aspartate, asparagine, glutamate or glutamine;
The residue corresponding to X279 is leucine, isoleucine, valine, alanine, glycine, phenylalanine, tyrosine, or tryptophan;
The residue corresponding to X281 is alanine, valine, isoleucine, leucine, asparagine, glutamine, serine or threonine;
The residue corresponding to X282 is histidine, aspartate, or glutamate;
The residue corresponding to X293 is aspartate, cysteine, lysine, phenylalanine or tyrosine;
The residue corresponding to X297 is arginine, lysine, phenylalanine, tyrosine or tryptophan;
The residue corresponding to X308 is leucine, isoleucine, valine, alanine, arginine, lysine, aspartate, or glutamate;
The residue corresponding to X337 is tyrosine, phenylalanine, tryptophan, lysine or arginine;
The residue corresponding to X345 is leucine, isoleucine, valine, alanine, arginine, or lysine;
The residue corresponding to X350 is asparagine, glutamine, serine, threonine, cysteine, or alanine;
The residue corresponding to X355 is phenylalanine, tryptophan, tyrosine, serine, threonine or cysteine.

9. Engineered benzaldehyde dehydrogenase of claim 1 that is at least 90% identical to the polypeptide given in SEQ ID 26, derived from the polypeptide sequence mentioned in the SEQ ID 25, which is the gene product of the “C” gene as given by SEQ ID 3 and that includes the feature of residue corresponding to X105 is R.

10. The engineered polypeptide of claim 9 comprising a polypeptide that is 90% identical to any of the amino acid sequences given in SEQ ID 27-33 and wherein the amino acid sequences additionally include at least one or more of the following features:
The residue corresponding to X9 is isoleucine, arginine, leucine, valine, alanine, lysine or histidine;
The residue corresponding to X10 is tryptophan, cysteine, serine, threonine, alanine, glycine, phenylalanine, valine, tyrosine or methionine;
The residue corresponding to X14 is valine, cysteine, isoleucine, leucine, alanine, serine, threonine, glycine, or methionine;
The residue corresponding to X18 is asparagine, glycine, alanine, serine, threonine, glutamine, or aspartate;
The residue corresponding to X26 is valine, glutamate, asparagine, glycine, alanine, serine, threonine, glutamine, glutamate, or aspartate;
The residue corresponding to X28 is asparagine, glutamate, asparagine, glycine, alanine, serine, threonine, glutamine, glutamate, or aspartate;
The residue corresponding to X37 is isoleucine, arginine, leucine, valine, alanine, lysine or histidine;
The residue corresponding to X40 is isoleucine, lysine, leucine, valine, alanine, arginine or histidine;
The residue corresponding to X42 is valine, cysteine, isoleucine, leucine, alanine, serine, threonine, glycine, or methionine;
The residue corresponding to X43 is isoleucine, arginine, leucine, valine, alanine, lysine or histidine;
The residue corresponding to X44 is alanine, cysteine, serine, threonine, glycine, proline or histidine;
The residue corresponding to X64 is alanine, cysteine, serine, threonine, glycine, proline or histidine;
The residue corresponding to X68 is tryptophan, cysteine, serine, threonine, alanine, glycine, phenylalanine, valine, tyrosine or methionine;
The residue corresponding to X87 is tryptophan, cysteine, serine, threonine, alanine, glycine, phenylalanine, valine, tyrosine, aspartate, glutamate, asparagine or methionine;
The residue corresponding to X122 is alanine, cysteine, serine, threonine, glycine, proline or histidine;
The residue corresponding to X129 is valine, glutamate, asparagine, glycine, alanine, serine, threonine, glutamine, glutamate, leucine, isoleucine or aspartate;
The residue corresponding to X140 is isoleucine, arginine, leucine, valine, alanine, lysine or histidine;
The residue corresponding to X148 is isoleucine, arginine, leucine, valine, alanine, lysine or histidine;
The residue corresponding to X155 is tryptophan, aspartate, glutamine, glycine, proline, serine, threonine, alanine, asparagine, glutamate, cysteine, phenylalanine, tyrosine or valine;
The residue corresponding to X161 is leucine, asparagine, aspartate, isoleucine, methionine, glutamine, glycine, proline, serine, threonine, alanine, glutamate, cysteine, or valine;
The residue corresponding to X173 is glycine, cysteine, serine, threonine, alanine, valine, or methionine;
The residue corresponding to X177 is isoleucine, aspartate, leucine, valine, alanine, glutamate or asparagine;
The residue corresponding to X178 is isoleucine, arginine, leucine, valine, alanine, lysine or histidine;
The residue corresponding to X190 is glycine, cysteine, serine, threonine, alanine, valine, or methionine;
The residue corresponding to X206 is isoleucine, aspartate, leucine, valine, alanine, glutamate or asparagine;
The residue corresponding to X209 is leucine, cysteine, isoleucine, valine, alanine, serine, threonine, glycine or proline
The residue corresponding to X218 is serine, threonine, alanine, lysine, glycine, valine, arginine, histidine or proline;
The residue corresponding to X225 is isoleucine, aspartate, leucine, valine, alanine, glutamate or asparagine;
The residue corresponding to X274 is serine, glutamate, aspartate, asparagine, threonine, glycine, valine, alanine or cysteine;
The residue corresponding to X317 is isoleucine, arginine, leucine, valine, alanine, lysine or histidine;
The residue corresponding to X323 is aspartate, glutamine, glutamate, asparagine, serine or threonine;
The residue corresponding to X352 is glutamine, arginine, asparagine, lysine, histidine, serine or cysteine;
The residue corresponding to X365 is aspartate, glutamine, glutamate, asparagine, serine or threonine;
The residue corresponding to X380 is lysine, phenylalanine, arginine, tryptophan, tyrosine, or histidine;
The residue corresponding to X381 is serine, glutamine, threonine, cysteine, asparagine or aspartate;
The residue corresponding to X383 is isoleucine, cysteine, valine, methionine, histidine, leucine, alanine, serine or threonine;
The residue corresponding to X385 is glycine, histidine, methionine, proline, valine, alanine, cysteine, serine, threonine, or lysine;
The residue corresponding to X432 is serine, glutamine, threonine, cysteine, glycine, asparagine, glutamate or aspartate;
The residue corresponding to X436 is isoleucine, arginine, leucine, valine, alanine, lysine or histidine;
The residue corresponding to X443 is cysteine, leucine, phenylalanine, proline, serine, threonine, isoleucine, tyrosine, tryptophan, histidine, alanine or valine;
The residue corresponding to X449 is phenylalanine, aspartate, tyrosine, tryptophan, glutamate, asparagine, or glutamine;
The residue corresponding to X451 is glycine, arginine, lysine, alanine, histidine, serine or threonine;
The residue corresponding to X461 is phenylalanine, isoleucine, lysine, leucine, arginine, tyrosine, tryptophan, or valine;
The residue corresponding to X462 is glycine, asparagine, alanine, serine, threonine, glutamine, or aspartate;
The residue corresponding to X465 is alanine, glutamine, serine, asparagine, threonine; glycine or aspartate;
The residue corresponding to X472 is glutamine, glutamate, asparagine, aspartate, serine, threonine, or alanine;
The residue corresponding to X475 is lysine, phenylalanine, arginine, tryptophan, tyrosine, or histidine;
The residue corresponding to X476 is isoleucine, aspartate, leucine, valine, alanine, glutamate or asparagine;
The residue corresponding to X483 is alanine, glutamate, tyrosine, phenylalanine, tryptophan, serine, threonine, valine or glycine;
The residue corresponding to X484 is asparagine, arginine, aspartate, glutamine, glutamate, lysine, histidine, serine, threonine or tyrosine.

11. The engineered benzyl alcohol dehydrogenase of claim 1 that is at least 90% identical to the polypeptide given in SEQ ID 35, derived from the polypeptide sequence mentioned in the SEQ ID 34, which is the gene product of the “B” gene as given by SEQ ID 4 and that includes the feature of the residue corresponding to X72 is Arg.

12. The engineered polypeptide of claim 11 comprising a polypeptide that is 90% identical to any of the amino acid sequences given in SEQ ID 36-40 and wherein the amino acid sequences additionally include at least one or more of the following features:
The residue corresponding to X23 is, asparagine, arginine, lysine, glutamine, or aspartate;
The residue corresponding to X27 is, glutamate, alanine, glycine, serine, threonine, aspartate, asparagine, glutamine, or valine;
The residue corresponding to X36 is, alanine, arginine, serine, threonine, glycine, lysine, or valine;
The residue corresponding to X38 is, alanine, arginine, serine, threonine, glycine, lysine, or valine;
The residue corresponding to X45 is, valine, arginine, tryptophan, lysine, leucine, isoleucine, phenylalanine, or tyrosine;
The residue corresponding to X46 is, cysteine, arginine, tyrosine, tryptophan, phenylalanine, serine, threonine, or lysine;
The residue corresponding to X52 is, proline, glycine, isoleucine, threonine, serine, leucine, valine, or alanine;
The residue corresponding to X73 is, alanine, histidine, serine, threonine, glycine, or valine;
The residue corresponding to X75 is, lysine, glutamate, arginine, aspartate, asparagine, glutamine, or histidine;
The residue corresponding to X99 is, glycine, aspartate, serine, threonine, alanine, valine, glutamate, or asparagine;
The residue corresponding to X112 is, phenylalanine, tyrosine, tryptophan, or histidine;
The residue corresponding to X118 is, threonine, arginine, serine, lysine, or alanine;
The residue corresponding to X123 is, isoleucine, histidine, leucine, valine, phenylalanine, tryptophan, tyrosine, or alanine;
The residue corresponding to X124 is, histidine, aspartate, glutamate, lysine, arginine, or asparagine;
The residue corresponding to X126 is, histidine, alanine, cysteine, serine, threonine, glycine, or methionine;
The residue corresponding to X127 is, glutamine, alanine, aspartate, asparagine, glycine, glutamate, serine, or threonine;
The residue corresponding to X128 is, glycine, leucine, lysine, alanine, valine, isoleucine, serine, or threonine;
The residue corresponding to X132 is, glycine, serine, threonine, alanine, cysteine, or valine;
The residue corresponding to X133 is, glycine, serine, threonine, alanine, cysteine, or valine;
The residue corresponding to X137 is, glycine, serine, threonine, alanine, cysteine, valine, asparagine, aspartate, glutamine, or glutamate;
The residue corresponding to X138 is, glycine, serine, threonine, alanine, cysteine, valine, asparagine, aspartate, glutamine, or glutamate;
The residue corresponding to X175 is, glycine, serine, threonine, alanine, cysteine, or valine;
The residue corresponding to X179 is, leucine, glutamate, isoleucine, valine, aspartate, asparagine, glutamine, or alanine;
The residue corresponding to X189 is, alanine, glutamate, valine, aspartate, asparagine, glutamine, serine, or threonine;
The residue corresponding to X204 is, methionine, aspartate, asparagine, glutamine, glutamate, lysine, or alanine;
The residue corresponding to X205 is, glycine, serine, threonine, alanine, cysteine, or valine;
The residue corresponding to X206 is, alanine, lysine, arginine, serine, threonine, or valine;
The residue corresponding to X207 is, glycine, serine, threonine, alanine, cysteine, or valine;
The residue corresponding to X211 is, glycine, serine, threonine, alanine, cysteine, valine, asparagine, aspartate, glutamine, or glutamate;
The residue corresponding to X213 is, glycine, serine, threonine, alanine, cysteine, valine, asparagine, aspartate, glutamine, or glutamate;
The residue corresponding to X224 is, leucine, cysteine, serine, threonine, isoleucine, methionine, valine, or alanine;
The residue corresponding to X227 is, glycine, serine, threonine, alanine, cysteine, or valine;
The residue corresponding to X230 is, leucine, arginine, isoleucine, valine, or lysine;
The residue corresponding to X231 is, glycine, serine, threonine, alanine, cysteine, or valine;
The residue corresponding to X232 is, glycine, serine, threonine, alanine, cysteine, or valine;
The residue corresponding to X235 is, leucine, cysteine, serine, threonine, isoleucine, methionine, valine, or alanine;
The residue corresponding to X240 is, alanine, lysine, arginine, serine, threonine, or valine;
The residue corresponding to X241 is, lysine, glutamate, arginine, aspartate, asparagine, glutamine, or histidine;
The residue corresponding to X251 is, phenylalanine, arginine, tyrosine, lysine, tryptophan, or histidine;
The residue corresponding to X252 is, alanine, glutamate, isoleucine, leucine, valine, aspartate, asparagine, or glutamine;
The residue corresponding to X253 is, aspartate, phenylalanine, glutamate, tyrosine, asparagine, tryptophan, glutamine, or histidine;
The residue corresponding to X256 is, proline, isoleucine, lysine, valine, leucine, alanine, arginine, or glycine;
The residue corresponding to X275 is, glycine, serine, threonine, alanine, cysteine, or valine;
The residue corresponding to X279 is, glycine, serine, threonine, alanine, cysteine, or valine;
The residue corresponding to X286 is, alanine, asparagine, histidine, threonine, serine, aspartate, or valine;
The residue corresponding to X301 is, leucine, histidine, tyrosine, isoleucine, phenylalanine, valine, or tryptophan;
The residue corresponding to X310 is, leucine, histidine, tyrosine, isoleucine, phenylalanine, valine, or tryptophan;
The residue corresponding to X311 is, aspartate, phenylalanine, glutamate, tyrosine, asparagine, tryptophan, glutamine, or histidine;
The residue corresponding to X313 is, glutamine, glutamate, asparagine, aspartate, serine, or threonine;
The residue corresponding to X315 is, isoleucine, arginine, leucine, lysine, valine, or histidine;
The residue corresponding to X326 is, leucine, arginine, cysteine, isoleucine, lysine, serine, valine, alanine, or threonine;
The residue corresponding to X332 is, phenylalanine, cysteine, tryptophan, serine, threonine, tyrosine, or alanine;
The residue corresponding to X350 is, glycine, serine, threonine, alanine, cysteine, valine, asparagine, aspartate, glutamine, or glutamate.

13. The whole cell catalysis of claim 1 wherein
a. the cell membrane permeability of the recombinant organism for increased substrate diffusion is increased using detergents like Tween 80 (Tw80) and Triton X-100 (TX100)
b. the external oxygen supply was provided to improve the activity of the whole cell catalysts.
c. The transforming recombinant host cell is immobilized on a suitable matrix for reusability.

14. The engineering method of claim 1 involves the computational method of the pLDDT-based protein optimization protocol (P-POP) wherein,
a. A 3D structure of the enzyme is studied, and hotspots are derived from Rational-based approach and particular residues with lower pLDDT scores.
b. An evolutionary analysis using a phylogeny-based approach is used to determine the substitution mutations for the hotspots and these substitutions are validated based on a pLDDT-scoring method, wherein residues with lower pLDDT scores are considered as hotspots for engineering.
c. Evolutionary analysis is used to determine the probability (Pxᵢ) of each amino acid (x) to occur at position P₁ as a function of the frequency of amino acid x occurring at position i (fₓᵢ) and the total number of sequences studied (N).
d. For these positions, evolutionary analysis and pLDDT score validation is used to determine best probable substitutions wherein an improvement in the pLDDT-score post mutation when compared to the pLDDT-score of the same position in the wild-type protein is desired.
e. Top scoring variants are validated in vitro, and the results are used to further refine the hotspot selection, and substitution protocols.
f. The final variants are selected through parameter optimization of the screened variants.


SEQUENCE LISTING (40 sequences)

SEQ ID NO: 1 - DNA, 1110 bp, synthetic construct
  source 1..1110: mol_type = other DNA
atggataccctgcgttactacctgattccggttgttaccgcttgcggtctgattggcttttattacggcggctattgggtttggttaggcgcagcaacctttccggcattaatggttctggacgtcattctgccgaaagattttagcgcgcgcaaagttagcccgtttttcgcagatctgacccagtatctgcaactgccgctgatgatcggtttatacggcctgctggtttttggcgttgaaaacggtcgcatcgaattaagcgaaccgctgcaagttgcaggttgtattctgagcctggcttggttatctggcgttccgaccttaccggtttctcacgaactgatgcatcgtcgtcactggttaccgcgtaaaatggctcaactgctggcaatgttttacggcgatccgaatcgcgatattgcgcacgttaacacccaccatctgtacctggataccccgttagattctgataccccgtatcgcggccagaccatttacagctttgtcattagcgcgaccgttggctccgttaaagatgcgattaaaatcgaagcggaaaccctgcgtcgtaaaggtcaaagtccgtggaatctgagcaacaaaacctaccagtacgtcgcactgctgctggcgttaccgggtttagttagctatctgggcggtccggcattaggtttagttaccattgcgagcatgatcattgccaaaggcattgtggagggcttcaactatttccagcattacggcctggttcgcgatttagatcagccgattctgctgcatcacgcctggaatcacatgggcaccatcgttcgtccgttaggttgcgaaatcaccaaccacatcaaccaccacatcgacggctatacccgtttctacgaactgcgtccggaaaaagaagctccgcaaatgccgagtctgttcgtttgctttctgctgggcttaattccgccgttatggtttgcgctgattgcgaaaccgaaactgcgcgattgggatcaacgttacgctaccccgggtgaacgcgagttagcaatggcagcaaataaaaaagccggttggccgctgtggtgcgaaagcgaattaggtcgcgttgcgagcatttaa

SEQ ID NO: 2 - DNA, 1053 bp, Synthetic construct
  source 1..1053: mol_type = other DNA
atgaacgagttcttcaaaaagattagcggcctgtttgttccgccaccagaaagtaccgttagcgttcgcggtcagggctttcagtttaaagtaccacgcggtcagaccattctggaatctgcactgcatcaaggtattgcctttccgcatgattgtaaagttggtagctgcggtacctgcaaatacaaactgatcagcggccgtgttaacgaactgacctcttctgcaatgggtctgtctggcgatctgtatcagagcggttatcgtctgggttgtcagtgtattccgaaagaggacctggagattgaactggataccgttctgggtcaggcactggttccaattgaaaccagcgcactgatcagcaaacagaaacgtctggcgcacgatatcgtcgaaatggaagtcgtcccggacaaacagattgcgttttatccgggtcagtacgctgacgttgaatgcgcagagtgttctgcagttcgtagctatagtttttctgcaccgccgcaaccagatggtagcctgagttttcacgtacgtctggttccaggcggcgtgtttagcggttggctgtttggcggcgatcgtaccggcgcaaccctgaccctgcgcgcaccatacggtcaatttggtctgcacgagtctaacgctacgatggtgtgcgttgcaggcggtaccggtctggcaccaattaaatgcgtcctgcagagcatgacccaggcacaacgcgaacgcgacgttctgctgtttttcggcgcacgtcaacaacgcgatctgtattgcctggacgaaatcgaagcactgcaactggattggggcggtcgttttgaactgattccggttctgagcgaagaaagcagtaccagtagctggaaaggtaaacgcggtatggtcaccgagtacttcaaagaatacctgaccggccagccatacgaaggttatctgtgcggtccaccaccaatggttgacgcagcagaaaccgaactggttcgtctgggcgttgcacgcgaactggtttttgcggatcgtttttataaccgtccgccatgttaa

SEQ ID NO: 3 - DNA, 1464 bp, synthetic construct
  source 1..1464: mol_type = other DNA
atgcgtgaaaccaaagagcagccgatttggtacggcaaagtcttcagcagcaactgggttgaagcacgtggcggcgttgcaaacgtagttgatccgagcaacggcgatattctgggtattaccggcgttgcaaacggcgaagacgttgatgcagcagtaaacgcagcaaaacgcgcacaaaaagagtgggcagcaattccgttcagcgaacgtgcagcaattgttcgtaaagcggcagaaaaactgaaagagcgcgagtacgagttcgcagattggaacgttcgcgagtgcggcgcaattcgtccgaaaggtctgtgggaagcaggcattgcttacgaacagatgcatcaagcagcaggtctggcaagtctgccgaacggtaccctgtttccgtctgctgttccgggtcgtatgaatctgtgtcaacgcgttccggtaggcgttgttggtgttattgcaccgtggaacttcccgctgtttctggcaatgcgttctgttgcaccggcactggcactgggtaacgcggttattctgaaaccggatctgcaaaccgcagttaccggcggcgcactgattgcggaaatttttagcgacgcgggtatgccggacggcgttctgcacgttctgccgggcggtgcagacgttggcgaaagtatggttgcaaactccggcatcaacatgatcagcttcaccggtagtacccaggtaggtcgtctgattggcgaaaaatgcggccgtatgctgaaaaaagttgcactggaactgggcggtaacaacgttcacattgttctgccggacgcagatctggaaggcgcagttagttgcgcagcttggggtacctttctgcatcagggtcaggtttgcatggcagctggtcgtcatctggttcatcgcgacgttgcacaacaatacgcggaaaaactggcactgcgcgcaaaaaatctggttgttggcgatccgaacagcgatcaagttcatctgggtccgctgatcaacgagaaacaggtcgttcgcgttcacgcactggttgaatctgctcaacgcgcaggtgcacaagttctggcaggcggtacctatcaagatcgctattaccaggccaccgtcattatggacgtcaaaccggagatggaggtcttcaaaagcgaaatctttggtccggttgcaccgattaccgtgtttgatagcatcgaagaagcgatcgaactggcaaattgcagcgaatacggtctggcagcaagtattcatacccgcgcactggcaaccggtctggatattgcaaaacgcctgaacaccggcatggttcatatcaacgatcagccgatcaattgcgaaccgcacgttccgtttggcggtatgggcgcatctggttctggcggtcgttttggcggtccggcaagcattgaagaatttacccagagtcagtggatcagcatggtggaaaaaccggcgaactatccgttttaa

SEQ ID NO: 4 - DNA, 1101 bp, synthetic construct
  source 1..1101: mol_type = other DNA
atggaaatcaaagcggcgatcgtccgtcagaaaaacggtccgtttctgctggaacacgttgcactgaacgaaccggcagaagatcaagttctggttcgtctggttgctaccggtctgtgtcataccgatctggtttgccgcgatcaacattatccggttccgctgccgatggtatttggtcacgaaggcgcaggcgtagttgaacgcgttggtagcgcggttaaaaaagttcagccgggcgatcacgttgttctgaccttttatacctgcggtagttgcgacgcttgtctgtctggcgatccgaccagttgcgcaaatagttttggcccgaactttatgggtcgttctgttaccggcgaatgtaccattcacgatcatcagggcgcagaagttggcgcaagtttctttggccagagcagctttgcaacctacgcactgagctatgaacgcaacaccgtcaaagtcaccaaagacgttccgctggaactgctgggtccgctgggttgcggtattcaaaccggcgcaggttctgttctgaatgcactgaatccgccggcaggttctgcaattgcaatttttggcgctggcgcagttggtctgtctgcagttatggcagcagttgttgcaggttgtaccaccattatcgcggttgatgtcaaagaaaaccgtctggaactggcaagcgaactgggcgcaacccatattattaacccggcagcgaacgatccgattgaagcgatcaaagagatttttgcagacggcgttccgtacgttctggaaacctctggtctgccggcagttctgacccaggcaattctgtcttctgcgattggtggcgaaattggtattgtaggcgctccgccgatgggcgcaaccgttccggttgatattaacttcctgctgttcaaccgtaaactgcgcggtattgtcgaaggccagagcatcagcgatatcttcattccgcgtctggtcgaactgtatcgtcagggcaaattcccgttcgacaaactgatcaaattctacccgttcgacgaaatcaaccgcgcagcagaagatagcgaaaaaggcgttaccctgaaaccggttctgcgtattggttaa

SEQ ID NO: 5 - DNA, 1131 bp, synthetic construct
  source 1..1131: mol_type = other DNA
atgtgggaatacatcaagtactacttcgcacccctggtacaagtctttgcgattctggggttctattggggtggtcattacacttggattgcaattggggcttttccggccattgccatccttgacatgctgctgccgctcgatctatctgagcgccgaatgaagaaccatttctgggcttacttgccgatctgggcttcgacgctgttattgccgttcatgtacttcggcttcgcgtggtcggtagcaaataacgatctgaccggattgcaaatggcagccggtgtgctggggttggcatggctttcggttgtgcctggcgttcctgcaacccacgagttgtatcactcgcgcgggcggttggctcgctttgttgggcgctatggccagatcgtatttctcgatgtcatgcgcatggaagtgcacgtagtcggccaccaccgcgacgtggggacggctgaggacattgatactgcggcacgcggtatgaatctgtacagattcgtcgttcgtgcagtggtggagtccgtccgctgggagctgaagctggatgcagacaatctggagaaacgcggttacggtcgctattcgatccgtcactcggtctggcgcgcgctgctggccattgtagtgttcctggggtgcatctatgccattggcggtgggatggcggtgggtctttgttcgatctcgatgatcatcgctcgtttttgggttgaggcattcaactactaccagcattacggccaagtccgtttggtcggcatgcccatcgaaaagcggcacgtctggaatcactttggaacgctctcacgcttgtatgcgtttgaaatcaccaaccacgcagaccaccatctcaactcctacatcccatattacaagctggtgccggatcgtgaggcgatcatcataccaagcatcatcgcatgcttcctgtcgggcttcattccacccctttggtatcgagcgatcatcaaacctgcgctcaaacgctgggacaacgaatatgcatcaccggccgagcggcgccttgcgcaggagcagaatcgtgaggcgggatgggatgattggttcaatgatcagggagggcgtggcagtcagcctgatggtccttccttggctcgcggctga

SEQ ID NO: 6 - DNA, 1050 bp, synthetic construct
  source 1..1050: mol_type = other DNA
atgagaagtttctttcaatccatcttcggcaaggcagtgcagaaacaactgcgaatcctgcctcaggacataacgatctcgcttaatccggggcaaacgctgctggaggctgtgttggccaatggtattgcctatcctcacgactgtacggttggtacctgcgcctcttgcaagacgcgcctgaagcaggggcgtgttcgagaggcaactccgtttggttacaccttgtcaaaggctgagctggatgcgggctacattctggcctgccaagcgtttccgcgtgatgaactcaccgtagttgagattgacccgccgtcggccgatcagccggcaaccgagcagttcgctgccactattgtagcgacggagccgctcactcacgacattctcaaggtagtgatccagtcggatcgaccagtgcactatctggccggccagtatgcgaacttgcgtgtcccgggttcctctcgcttccgctcttattcattcgccaatgcaccgcaacgcaaggggcagagcacccttgagttctatatccgcaaggtgccaggaggcgaattcaccgaagccttgttccgtggcgagttggatggtcggccactggaaatggaggctccccaaggtaccttccacttgcatggcggtgatgcgccgatggtgtgtatcgctggaggcagtggtttggcaccactgataagtatcctacagcacgcgcgagcaaatcgtatcaagcgagactgtacgttgttgtttggcgcacgcactcaggatgatctatatcaactcgatataatcagcaatatcgctgccaactggcaaggtgattttcgttttattccggtgctttcgcatgaacaggaatgcagtaactggaccggcgcacgtggccttgtgaccgagcatattgctgctgatttctgcgagggggcagagggttatctctgtggacctccaccgatgatagatgccgccatctctcgtcttgcggagcaaggcataccgttggaaagagtcttttatgacaagtttacggacggccggaactcctaa

SEQ ID NO: 7 - DNA, 13 bp, synthetic construct
  source 1..13: mol_type = other DNA
aggaggcataatt

SEQ ID NO: 8 - DNA, 10 bp, synthetic construct
  source 1..10: mol_type = other DNA
aggtggatat

SEQ ID NO: 9 - DNA, 16 bp, synthetic construct
  source 1..16: mol_type = other DNA
tgagcgcgatgggttt

SEQ ID NO: 10 - DNA, 12 bp, synthetic construct
  source 1..12: mol_type = other DNA
ataaagagggag

SEQ ID NO: 11 - DNA, 16 bp, synthetic construct
  source 1..16: mol_type = other DNA
tgagcgcgattggttt

SEQ ID NO: 12 - protein, 369 aa, Pseudomonas putida
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDIAHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 13 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 14 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFQYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 15 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFRHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 16 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIRLSEPLQVAGCILSLAWLSGVPTLPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 17 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSRPLQVAGCILSLAWLSGVPTLPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 18 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLQCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 19 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEGTNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 20 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTHPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 21 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLERKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 22 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGANYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 23 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLRVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIVEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 24 - protein, 369 aa, synthetic construct
MDTLRYYLIPVVTACGLIGFYYGGYWVWLGAATFPALMVLDVILPKDFSARKVSPFFADLTQYLQLPLMIGLYGLLVFGVENGRIELSEPLQVAGCILSLAWLSGVPTLPVSHELMHRRHWLPRKMAQLLAMFYGDPNRDITHVNTHHLYLDTPLDSDTPYRGQTIYSFVISATVGSVKDAIKIEAETLRRKGQSPWNLSNKTYQYVALLLALPGLVSYLGGPALGLVTIASMIIAKGIAEGFNYFQHYGLVRDLDQPILLHHAWNHMGTIVRPLGCEITNHINHHIDGYTRFYELRPEKEAPQMPSLFVCFLLGLIPPLWFALIAKPKLRDWDQRYATPGERELAMAANKKAGWPLWCESELGRVASI

SEQ ID NO: 25 - protein, 487 aa, Pseudomonas putida
MRETKEQPIWYGKVFSSNWVEARGGVANVVDPSNGDILGITGVANGEDVDAAVNAAKRAQKEWAAIPFSERAAIVRKAAEKLKEREYEFADWNVRECGAIRPKGLWEAGIAYEQMHQAAGLASLPNGTLFPSAVPGRMNLCQRVPVGVVGVIAPWNFPLFLAMRSVAPALALGNAVILKPDLQTAVTGGALIAEIFSDAGMPDGVLHVLPGGADVGESMVANSGINMISFTGSTQVGRLIGEKCGRMLKKVALELGGNNVHIVLPDADLEGAVSCAAWGTFLHQGQVCMAAGRHLVHRDVAQQYAEKLALRAKNLVVGDPNSDQVHLGPLINEKQVVRVHALVESAQRAGAQVLAGGTYQDRYYQATVIMDVKPEMEVFKSEIFGPVAPITVFDSIEEAIELANCSEYGLAASIHTRALATGLDIAKRLNTGMVHINDQPINCEPHVPFGGMGASGSGGRFGGPASIEEFTQSQWISMVEKPANYPF

SEQ ID NO: 26 - protein, 487 aa, synthetic construct
MRETKEQPIWYGKVFSSNWVEARGGVANVVDPSNGDILGITGVANGEDVDAAVNAAKRAQKEWAAIPFSERAAIVRKAAEKLKEREYEFADWNVRECGAIRPKGRWEAGIAYEQMHQAAGLASLPNGTLFPSAVPGRMNLCQRVPVGVVGVIAPWNFPLFLAMRSVAPALALGNAVILKPDLQTAVTGGALIAEIFSDAGMPDGVLHVLPGGADVGESMVANSGINMISFTGSTQVGRLIGEKCGRMLKKVALELGGNNVHIVLPDADLEGAVSCAAWGTFLHQGQVCMAAGRHLVHRDVAQQYAEKLALRAKNLVVGDPNSDQVHLGPLINEKQVVRVHALVESAQRAGAQVLAGGTYQDRYYQATVIMDVKPEMEVFKSEIFGPVAPITVFDSIEEAIELANCSEYGLAASIHTRALATGLDIAKRLNTGMVHINDQPINCEPHVPFGGMGASGSGGRFGGPASIEEFTQSQWISMVEKPANYPF

SEQ ID NO: 27 - protein, 487 aa, synthetic construct
MRETKEQPLWYGKVFSSNWVEARGGVANVVDPSNGDILGITGVANGEDVDAAVNAAKRAQKEWAAIPFSERAAIVRKAAEKLKEREYEFADWNVRECGAIRPKGRWEAGIAYEQMHQAAGLASLPNGTLFPSAVPGRMNLCQRVPVGVVGVIAPWNFPLFLAMRSVAPALALGNAVILKPDLQTAVTGGALIAEIFSDAGMPDGVLHVLPGGADVGESMVANSGINMISFTGSTQVGRLIGEKCGRMLKKVALELGGNNVHIVLPDADLEGAVSCAAWGTFLHQGQVCMAAGRHLVHRDVAQQYAEKLALRAKNLVVGDPNSDQVHLGPLINEKQVVRVHALVESAQRAGAQVLAGGTYQDRYYQATVIMDVKPEMEVFKSEIFGPVAPITVFDSIEEAIELANCSEYGLAASIHTRALATGLDIAKRLNTGMVHINDQPINCEPHVPFGGMGASGSGGRFGGPASIEEFTQSQWISMVEKPANYPF

SEQ ID NO: 28 - protein, 487 aa, synthetic construct
MRETKEQPIWYGKVFSSNWVESRGGVANVVDPSNGDILGITGVANGEDVDAAVNAAKRAQKEWAAIPFSERAAIVRKAAEKLKEREYEFADWNVRECGAIRPKGRWEAGIAYEQMHQAAGLASLPNGTLFPSAVPGRMNLCQRVPVGVVGVIAPWNFPLFLAMRSVAPALALGNAVILKPDLQTAVTGGALIAEIFSDAGMPDGVLHVLPGGADVGESMVANSGINMISFTGSTQVGRLIGEKCGRMLKKVALELGGNNVHIVLPDADLEGAVSCAAWGTFLHQGQVCMAAGRHLVHRDVAQQYAEKLALRAKNLVVGDPNSDQVHLGPLINEKQVVRVHALVESAQRAGAQVLAGGTYQDRYYQATVIMDVKPEMEVFKSEIFGPVAPITVFDSIEEAIELANCSEYGLAASIHTRALATGLDIAKRLNTGMVHINDQPINCEPHVPFGGMGASGSGGRFGGPASIEEFTQSQWISMVEKPANYPF

SEQ ID NO: 29 - protein, 487 aa, synthetic construct
MRETKEQPIWYGKVFSSNWVEARGGVANVVDPSNGDILGITGVANGEDVDAAVNAAKRAQKEWAAIPFSERAAIVRKAAEKLKEREYEFADWNVRECGAIRPKGRWEAGIAYEQMHQAAGLASLPNGTLFPSAVPGRMNLCQRVPVGVVGVIAPWNFPLFLAMRSVAPALALGNAVILKPDLQTAVTGGALIAEIFSDAGMPDGVLHVLPGGADVGESMVANSGINMISFTGSTQVGRLIGEKCGRMLKKVALELGGNNVHIVLPDADLEGAVSCAAWGTFLHQGQVCMAAGRHLVHRDVAQQYAEKLALRAKNLVVGDPNSDQVHLGPLINEKQVVRVHALVESAQRAGAQVLAGGTYQDRYYQATVIMDVKPEMEVFKSEIFGPVAPITVFDSIEEAIELANCSEYGLAASIHTRALATGLDIAKRLNTGMVHINDQPINCEPHVPFGGMGASGSGGRFGGPASIEEFTESQWISMVEKPANYPF

SEQ ID NO: 30 - protein, 487 aa, synthetic construct
MRETKEQPIWYGKVFSSNWVEARGGVANVVDPSNGDILGITGRANGEDVDAAVNAAKRAQKEWAAIPFSERAAIVRKAAEKLKEREYEFADWNVRECGAIRPKGRWEAGIAYEQMHQAAGLASLPNGTLFPSAVPGRMNLCQRVPVGVVGVIAPWNFPLFLAMRSVAPALALGNAVILKPDLQTAVTGGALIAEIFSDAGMPDGVLHVLPGGADVGESMVANSGINMISFTGSTQVGRLIGEKCGRMLKKVALELGGNNVHIVLPDADLEGAVSCAAWGTFLHQGQVCMAAGRHLVHRDVAQQYAEKLALRAKNLVVGDPNSDQVHLGPLINEKQVVRVHALVESAQRAGAQVLAGGTYQDRYYQATVIMDVKPEMEVFKSEIFGPVAPITVFDSIEEAIELANCSEYGLAASIHTRALATGLDIAKRLNTGMVHINDQPINCEPHVPFGGMGASGSGGRFGGPASIEEFTQSQWISMVEKPANYPF

SEQ ID NO: 31 - protein, 487 aa, synthetic construct
MRETKEQPIWYGKVFSSNWVEARGGVANVVDPSNGDILGITGVANGEDVDAAVNAAKRAQKEWAAIPFSERAAIVRKAAEKLKEREYEFADWNVRECGAIRPKGRWEAGIAYEQMHQAAGLASLPNGTLFPSAVPGRMNLCQRVPVGVVGVIAPWNFPLFLAMRSVAPALALGNAVILKPDLQTAVTGGALIAEIFSDAGMPDGVLHVLPGGADVGEKMVANSGINMISFTGSTQVGRLIGEKCGRMLKKVALELGGNNVHIVLPDADLEGAVSCAAWGTFLHQGQVCMAAGRHLVHRDVAQQYAEKLALRAKNLVVGDPNSDQVHLGPLINEKQVVRVHALVESAQRAGAQVLAGGTYQDRYYQATVIMDVKPEMEVFKSEIFGPVAPITVFDSIEEAIELANCSEYGLAASIHTRALATGLDIAKRLNTGMVHINDQPINCEPHVPFGGMGASGSGGRFGGPASIEEFTQSQWISMVEKPANYPF

SEQ ID NO: 32 - protein, 487 aa, synthetic construct
MRETKEQPIWYGKVFSSNWVEARGGVANVVDPSNGDILGITGVANGEDVDAAVNAAKRAQKEWAAIPFSERAAIVRKAAEKLKEREYEFADWNVRECGAIRPKGRWEAGIAYEQMHQAAGLASLPNGTLFPSAVPGRMNLCQRVPVGVVGVIAPWNFPLFLAMRSVAPALALGNAVILKPDLQTAVTGGALIAEIFSDAGMPDGVLHVLPGGADVGESMVANSGDNMISFTGSTQVGRLIGEKCGRMLKKVALELGGNNVHIVLPDADLEGAVSCAAWGTFLHQGQVCMAAGRHLVHRDVAQQYAEKLALRAKNLVVGDPNSDQVHLGPLINEKQVVRVHALVESAQRAGAQVLAGGTYQDRYYQATVIMDVKPEMEVFKSEIFGPVAPITVFDSIEEAIELANCSEYGLAASIHTRALATGLDIAKRLNTGMVHINDQPINCEPHVPFGGMGASGSGGRFGGPASIEEFTQSQWISMVEKPANYPF

SEQ ID NO: 33 - protein, 487 aa, synthetic construct
MRETKEQPIWYGKVFSSNWVEARGGVANVVDPSNGDILGITGVANGEDVDAAVNAAKRAQKEWAAIPFSERAAIVRKAAEKLKEREYEFADWNVRECGAIRPKGRWEAGIAYEQMHQAAGLASLPNGTLFPSAVPGRMNLCQRVPVGVVGVIAPWNFPLFLAMRSVAPALALGNAVILKPDLQTAVTGGALIAEIFSDAGMPDGVLHVLPGGADVGESMVANSGINMISFTGSTQVGRLIGEKCGRMLKKVALELGGNNVHIVLPDADLEGAVSCAAWGTFLHQGQVCMAAGRHLVHRDVAQQYAEKLALRAKNLVVGDPNSDQVHLGPLINEKQVVRVHALVESAQRAGAQVLAGGTYQDRYYQATVIMDVKPEMEVFKSEIFGPVAPITVFDSIEEAIELANCSEYGLAASIHTRALATGLDIAKRLNTDMVHINDQPINCEPHVPFGGMGASGSGGRFGGPASIEEFTQSQWISMVEKPANYPF

SEQ ID NO: 34 - protein, 366 aa, Pseudomonas putida
MEIKAAIVRQKNGPFLLEHVALNEPAEDQVLVRLVATGLCHTDLVCRDQHYPVPLPMVFGHEGAGVVERVGSAVKKVQPGDHVVLTFYTCGSCDACLSGDPTSCANSFGPNFMGRSVTGECTIHDHQGAEVGASFFGQSSFATYALSYERNTVKVTKDVPLELLGPLGCGIQTGAGSVLNALNPPAGSAIAIFGAGAVGLSAVMAAVVAGCTTIIAVDVKENRLELASELGATHIINPAANDPIEAIKEIFADGVPYVLETSGLPAVLTQAILSSAIGGEIGIVGAPPMGATVPVDINFLLFNRKLRGIVEGQSISDIFIPRLVELYRQGKFPFDKLIKFYPFDEINRAAEDSEKGVTLKPVLRIG

SEQ ID NO: 35 - protein, 366 aa, synthetic construct
MEIKAAIVRQKNGPFLLEHVALNEPAEDQVLVRLVATGLCHTDLVCRDQHYPVPLPMVFGHEGAGVVERVGRAVKKVQPGDHVVLTFYTCGSCDACLSGDPTSCANSFGPNFMGRSVTGECTIHDHQGAEVGASFFGQSSFATYALSYERNTVKVTKDVPLELLGPLGCGIQTGAGSVLNALNPPAGSAIAIFGAGAVGLSAVMAAVVAGCTTIIAVDVKENRLELASELGATHIINPAANDPIEAIKEIFADGVPYVLETSGLPAVLTQAILSSAIGGEIGIVGAPPMGATVPVDINFLLFNRKLRGIVEGQSISDIFIPRLVELYRQGKFPFDKLIKFYPFDEINRAAEDSEKGVTLKPVLRIG

SEQ ID NO: 36 - protein, 366 aa, synthetic construct
MEIKAAIVRQKNGPFLLEHVALNEPAGDQVLVRLVATGLCHTDLVCRDQHYIVPLPMVFGHEGAGVVERVGRAVKKVQPGDHVVLTFYTCGSCDACLSDDPTSCANSFGPNFMGRSVTGECTHHDHDGAEVGASFFDQSSFATYALSYERNTVKVTKDVPLELLGPLGCGIQTGAGSVENALNPPAGSAIAIFGAGAVGLSAVMAACVAGDTTIIAVDVKENRLELCSELGATHIINPAKNDPIEAIKEIFIFGVIYVLETSGLPAVLTQAILSSAIGGEIGIVGAPPMGATVPVDINFLYFNRKLRGIVEGQSISDIFIPRLVELYRQGKFPFDKLIKFYPFDEINRAAEDSEKGVTLKPVLRIG

SEQ ID NO: 37 - protein, 366 aa, synthetic construct
MEIKAAIVRQKNGPFLLEHVALNEPAEDQVLVRLVATGLCHTDLRRRDQHYPVPLIMVFGHEGAGVVERVGRAVKKVQPGDHVVLTFYTCGSCDACLSGDPTSCANSFGPNYMGRSVRGECTLDDHQGAEVSGSFFGSSSFATYALSYERNTVKVTKDVPLELLGPLGCGIQTGCGSVLNALNPPAGSAIAIFGAGAVGLSAVMAAVVAGCTTIIAVDVKENRCELASELGATHIINPAARDPIEAIKEIFADGVPYVLETSGLPAVLTQAILSSAIGGEIGIVGHPPMGATVPVDINFLLFNRKLRGIYEGQSISDIFIPRLVELYRQGKFPFDKLIKFYPFDEINRAAEDSEKGVTLKPVLRIG

SEQ ID NO: 38 - protein, 366 aa, synthetic construct
MEIKAAIVRQKNGPFLLEHVALREPAEDQVLVRLVATGLCHTDLWCRDQHYPVPLTMVFGHEGAGVVERVGRAVKKVQPGDHVVLTFYTCGSCDACLSGDPTSCANSFGPNFMGRSVTGECTIHDHQGAEVTSSFFGQSSFATYALSYERNTVKVTKDVPLELLGPLGCGIQTGAGSVLNALNPPAGSEIAIFGAGAVGLSAVMAKVVAGCTEIIAVDVKENRLELASELGATHCINPAANDPIEAIKEIRADGVVYVLETSGLPAVLTQAILSSAIGCEIGIVGAPPMGATVPVDINFLLFNRKLRGIVYGESISDIFIPRLVECYRQGKFPFDKLIKFYPFDEINRAAEDSEKGVTLKPVLRIG

SEQ ID NO: 39 - protein, 366 aa, synthetic construct
MEIKAAIVRQKNGPFLLEHVALNEPAADQVLVRLVRTRLCHTDLVCRDQHYGVPLPMVFGHEGAGVVERVGRAVEKVQPGDHVVLTFYTCGSCDACLSGDPTSCANSFGPNFMGRSVTGECTIHDCALAEVGASFFAQSSFATYALSYERNTVKVTKDVPLELLGPLGCGIQTGAGSVLNALNPPAGSAIAIFGAGAVGLSAVMAAVVAGCTTIIAVDVKENRLELASELGATHIINPAANDPIEAIKEIFEDGVPYVLETSGLPAVLTQAILSCAIGGEIGIVGNPPMGATVPVDINFLHFNRKLRGIVEGQSISDIFIPRLVELYRQGKCPFDKLIKFYPFDEINRAEEDSEKGVTLKPVLRIG

SEQ ID NO: 40 - protein, 366 aa, synthetic construct
MEIKAAIVRQKNGPFLLEHVALNEPAEDQVLVRLVATGLCHTDLVYRDQHYPVPLPMVFGHEGAGVVERVGRHVKKVQPGDHVVLTFYTCGSCDACLSGDPTSCANSFGPNFMGRSVTGECTIHDAQKAEVGASFFGQSSFATYALSYERNTVKVTKDVPLELLGPLGCGIQTGAGSVLNALNPPAGSAIAIFGAGAVGLSAVDCAVVAGCTTIIAVDVKENRLELASERCCTHIINPAANDPIEAIKEIFADGVKYVLETSGLPAVLTQAILSSAIGGEIGIVGTPPMGATVPVDINFLLFNRKLRGIWFGQSRSDIFIPRLVERYRQGKFPFDKLIKFYPFDEINRAAEDSEKGVTLKPVLRIG